(19) United States
(12) Patent Application Publication
(10) Pub. No.: US 2019/0328278 A1
(43) Pub. Date: Oct. 31, 2019

(54) SYSTEM AND METHOD FOR MONITORING RESPIRATION

(71) Applicant: 3M INNOVATIVE PROPERTIES COMPANY, SAINT PAUL, MN (US)

(72) Inventors: MATTHEW T. ZABEL, LAKE ELMO, MN (US); ROGER W. BARTON, AFTON, MN (US); SABER TAGHVAEEYAN, MAPLE GROVE, MN (US); JONATHAN B. ARTHUR, HUDSON, WI (US); CHRISTINE S. BELL, MINNEAPOLIS, MN (US); ARASH SANGARI, FRISCO, TX (US); NICHOLAS A. ASENDORF, SAINT PAUL, MN (US); TIMOTHY F. MUNOZ, EAST GREENWICH, MN (US); KEITH T. THORNLEY, CINCINNATI, OH (US)

(73) Assignee: 3M INNOVATIVE PROPERTIES COMPANY, SAINT PAUL, MN (US)

(21) Appl. No.: 16/475,149

(22) PCT Filed: Jan. 2, 2018
(86) PCT No.: PCT/US2018/012048

§ 371 Date: Jul. 1, 2019

Related U.S. Application Data
(60) Provisional application No. 62/441,955, filed on Jan. 3, 2017.

(51) Int. Cl. A61B 5/08; A61B 5/00; G16H 50/30

(52) U.S. Cl. CPC A61B 5/0816; A61B 5/742; A61B 5/7475; A61B 2560/0242; A61B 5/486; G16H 50/30; A61B 5/7275

(57) ABSTRACT

This disclosure provides systems and methods for monitoring respiratory parameters of a person (e.g., a user). In some examples, a system for respiratory monitoring includes a sensor and a computing device. The sensor monitors at least one respiratory related parameter. The computing device is connected to the sensor. The computing device includes a processor. The processor receives a feature vector having a plurality of features, each respective feature associated with a respective respiratory related parameter. The processor also receives from the sensor the at least one respective respiratory related parameter. The processor determines a first breathing state based on the feature vector.

This application claims the benefit of U.S. Provisional Application No. 62/441,955, filed Jan. 3, 2017, which is incorporated herein by reference in its entirety.

### TECHNICAL FIELD

This disclosure relates to systems and methods for monitoring biological signals, and, in particular, to systems and methods for monitoring respiration.

### BACKGROUND

Monitoring respiratory parameters of a person may be indicative of physiological states of the person, mental states of the person, or both. Physiological states and mental states of the person can include a stress level of the person, pulmonary disorders, breathing disorders, sleep disorders, and the like. Typically, respiratory monitoring is performed in a clinical setting with devices that may lack portability, may cause discomfort, and may require training to use.

### SUMMARY

The disclosure provides systems and methods for monitoring respiratory parameters of a person (e.g., a user). The system includes a sensor and a computing device. The sensor is a wearable device that detects respiratory parameters and generates respective outputs representative of the respective respiratory parameters. The respiratory parameters may include amplitudes, rates, and durations of inhalation, exhalation, or both. The computing device is communicatively coupled (e.g., wirelessly connected) to the sensor. The computing device may be portable, such as, for example, a mobile phone, to allow the user to remain ambulatory while monitoring the respiratory parameters of the user. The computing device includes a processor that receives the output from the sensor. In some examples, the processor determines an occurrence of a breathing event based on an analysis of the output of the sensor. The breathing event may include the detection of a breathing state such as, for instance, a smooth breathing state (e.g., periods of improved breathing relative to average breathing activity), a hard breathing state (e.g., periods of more labored breathing relative to average breathing activity), or the like. The computing device may store these breathing events and display, via a user interface, the breathing events to the user. In some examples, the computing device receives environmental information, user-provided information, or both and determines a trigger based on the received information. In some example, the trigger is associated with a respective breathing event. The computing device may store triggers and display, via a user interface, triggers to the user. In other examples, the computing device may cause the user interface to prompt the user to input triggers associated with a respective breathing event. The computing device may determine trends based on at least one of the sensor output, environmental information, user-provided information, breathing events, and triggers. The computing device may store trends and display, via a user interface, trends to the user. By storing and displaying breathing events, triggers, and trends, the systems and methods described herein may enable a user to identify triggers that cause improved breathing and worsened breathing.

In some examples, a method for respiratory monitoring includes receiving, by a computing device, a feature vector having a plurality of features, each respective feature associated with a respective respiratory related parameter. After receiving the feature vector, the method includes receiving, from a sensor, at least one respiratory related parameter associated with a respective feature of the feature vector. After receiving the at least one respiratory related parameter, the method includes detecting, by the computing device, a first breathing state based on the at least one respiratory related parameter received from the sensor.

In some examples, a method for respiratory monitoring includes receiving, from a sensor, at least one respiratory related parameter. After receiving the at least one respiratory related parameter, the method includes detecting, by a computing device, a first breathing state based on the at least one respiratory related parameter received from the sensor. After detecting the first breathing state, the method includes receiving, from a user interface, user input related to the first breathing state. After receiving the user input, the method includes modifying, by the computing device, the detection of one or more breathing states based the user input.

In some examples, a system for respiratory monitoring includes a sensor and a computing device. The sensor monitors at least one respiratory related parameter. The computing device is connected to the sensor. The computing device includes a processor. The processor receives a feature vector having a plurality of features, each respective feature associated with a respective respiratory related parameter. The processor also receives from the sensor the at least one respective respiratory related parameter. The processor determines a first breathing state based on the feature vector.

In some examples, a non-transitory computer-readable storage medium that stores computer system executable instructions that, when executed, may configure a processor to receive, by a computing device, a feature vector having a plurality of features, each respective feature associated with a respective respiratory related parameter. The non-transitory computer-readable storage medium may also store computer system-executable instructions that, when executed, may configure a processor to receive, from a sensor, at least one respiratory related parameter associated with a respective feature of the feature vector. The non-transitory computer-readable storage medium may also store computer system-executable instructions that, when executed, may configure a processor to detect a first breathing state based on the at least one respiratory related parameter received from the sensor.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a schematic and conceptual diagram illustrating an example respiration monitoring system for monitoring the respiration of a user.

FIG. 2 is a schematic and conceptual diagram illustrating an example user device that includes a computing device and a user interface.

FIG. 3 is a flow diagram of an example technique for notifying a user of an occurrence of a breathing event.

FIG. 4 is a flow diagram of an example technique for receiving, by a computing device, respiratory related parameters.

FIG. 5 is a flow diagram of an example technique for detecting a condition such as an individual's breathing state.

FIG. 6 is a flow diagram of an example technique for displaying a notification to a user via a display of a user interface.

FIG. 7 is a flow diagram of an example technique for adjusting a detection algorithm based on input from a user.

FIG. 8 is a flow diagram of an example technique for adjusting the algorithm based on user input in response to providing a user a notification.

FIG. 9 is a schematic and conceptual diagram illustrating an example display of a today view of a display of a respiration monitoring system.

FIG. 10 is a schematic and conceptual diagram illustrating an example display of a weather view of a display of a respiration monitoring system.

FIG. 11 is a schematic and conceptual diagram illustrating an example display of an air quality view of a display of a respiration monitoring system.

FIG. 12 is a schematic and conceptual diagram illustrating an example display of a breathing view of a display of a respiration monitoring system.

FIG. 13 is a schematic and conceptual diagram illustrating an example display of a notification on a today view of a display of a respiration monitoring system.

FIG. 14 is a schematic and conceptual diagram illustrating an example display of a breathing events view on a display of a respiration monitoring system.

FIG. 15 is a schematic and conceptual diagram illustrating an example display of an event details view on a display of a respiration monitoring system.

FIG. 16 is a schematic and conceptual diagram illustrating an example display of a trends view on a display of a respiration monitoring system.

FIG. 17 is a schematic and conceptual diagram illustrating an example display of a trigger trends view on a display of a respiration monitoring system.

FIG. 18 is a schematic and conceptual diagram illustrating an example display of a location trends view on a display of a respiration monitoring system.

FIG. 19 is a schematic and conceptual diagram illustrating an example display of a location trends view on a display of a respiration monitoring system.

FIG. 20 is a schematic and conceptual diagram illustrating an example display of an advice view on a display of a respiration monitoring system.

FIG. 21 is a schematic and conceptual diagram illustrating an example display of a determined trigger view on a display of a respiration monitoring system.

FIG. 22 is a schematic and conceptual diagram illustrating an example push notification view displayed on a display of a respiration monitoring system.

FIG. 23 is a schematic and conceptual diagram illustrating an example display of a location survey view.

FIG. 24 is a schematic and conceptual diagram illustrating an example display of a trigger survey view.

FIG. 25 is a schematic and conceptual diagram illustrating an example display of a symptoms survey view.

FIG. 26 is a schematic and conceptual diagram illustrating an example display of an event updated notification view.

### DETAILED DESCRIPTION

Respiratory monitoring systems may sense one or more respiratory parameters of a user. Respiratory parameters include any detectable parameter indicative of respiration, such as amplitudes, rates, and durations of inhalation, exhalation, or both, cycle time of a respiratory cycle, inspiratory pause, expiratory pause, tidal volume, ventilation flow rate, inspiratory flow, expiratory flow, pulse rate; oxygen-saturation, acoustic spectral density; and the like. Respiratory parameters may be indicative of physiological states of the user, such as sleep state, activity level, and the like, as well as pulmonary disorders, breathing disorders, sleep disorders, and the like; and mental states of the user, such as stress level and the like; or both. Systems and techniques of this disclosure provide a respiratory monitoring system that allows a user to remain ambulatory while tracking occurrences of breathing events, tracking triggers associated with respective occurrences of breathing events, tracking trends associated with occurrences of breathing events or triggers associated with respective occurrences of breathing events, and providing feedback to improve the quality of information provided to the user.

By allowing the user to remain ambulatory during respiratory data acquisition more respiratory data may be collected over time, at different locations, and during different activities compared to other respiratory monitoring systems. Because the respiratory data may be collected over time, the respiratory data may enable determination of an average respiration of the user (e.g., an average respiration amplitude, frequency, rate, or the like). The respiratory data may indicate an occurrence of a breathing event. A breathing event may include a change in the respiration of the user compared to an average respiration of the user. In some examples, a breathing event includes a transition into a hard breathing state, as may be indicated by an increase in at least one of an amplitude, a frequency, or a rate of breathing. In other examples, a breathing event includes detection of a smooth breathing state as may be indicated by a decrease in at least one of an amplitude, a frequency, or a rate of breathing. In this way, the systems and techniques of the disclosure enable a user to track occurrences of breathing events.

Occurrences of breathing events may be recorded while the user is performing daily tasks or irregular tasks. For example, some daily tasks, such as walking a flight of stairs, may trigger a hard breathing event, whereas other daily tasks, such as reading the newspaper, may trigger a smooth breathing event. Some intermittent tasks, such as walking to work on a day with high pollen count, may trigger a hard breathing event, whereas other intermittent tasks, such as attending a religious service, may trigger a smooth breathing event. The systems and techniques of the disclosure include notifying the user of breathing events in real time to improve the respiration of the user.

In some examples, the systems and methods of the disclosure enable the user to associate an occurrence of a breathing event with a trigger based on a daily task or irregular task. For example, a user may attribute an occurrence of a breathing event to a particular trigger. By associating triggers with occurrences of breathing events, the systems and methods of the disclosure allow a user to monitor or adjust behaviors to improve the respiration of the user. Further, the occurrences of breathing events and triggers may be stored over time to generate trends associated with types of breathing events and types of triggers. The trends may be displayed to the user to enable the user to adjust behaviors to improve the respiration of the user.

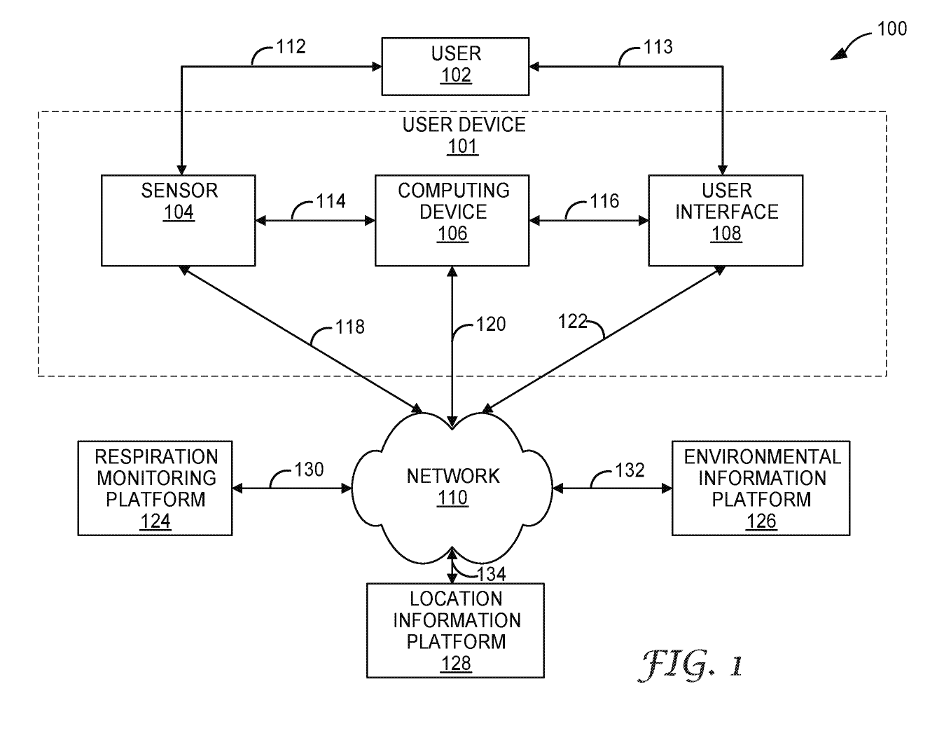

FIG. 1 is a schematic and conceptual diagram illustrating an example respiration monitoring system 100 for monitoring the respiration of a user 102. User 102 includes a human person that may be ambulatory. In other examples, user 102 may include a nonambulatory human or other ambulatory or nonambulatory animal species, such as domestic animals. In the example approach shown in FIG. 1, respiration monitoring system 100 includes user device 101. User device 101 includes a sensor 104, a computing device 106, and a user interface 108. In other examples, a sensor 104, a computing device 106, and a user interface 108 may define components of a separate devices. In some examples, respiration monitoring system 100 also includes a respiration monitoring platform 124, an environmental information platform 126, and a location information platform 128. Respiration monitoring system 100 may include additional components or fewer components.

Sensor 104 includes at least one sensor that detects at least one signal indicative of respiration characteristics of user 102 (e.g., respiration signals) at a sensor-user interface 112. For example, sensor 104 may be affixed to a portion of the body of user 102 (e.g., the torso of user 102) or affixed to a garment worn by user 102 (e.g., an elastic band, a waist band, a bra strap, or the like). In some examples, sensor 104 senses a movement of the torso of user 102 at sensor-user interface 112 that results from respiration of user 102. In this way, sensor 104 may detect the respiration of user 102 by respiratory inductance plethysmography (e.g., evaluating pulmonary ventilation by measuring the movement of the chest and abdominal wall). In other examples, sensor 104 detects respiration signals of user 102 by other means. For example, sensor 104 may include invasive or minimally invasive components, such as masks or mouthpieces coupled to the airway of user 102. Alternatively, or additionally, sensor 104 may include other suitable sensors, or combination of sensors, to detect respiration of user 102, such as strain gauge sensors, pressure sensors, accelerometers, gyroscopes, displacement sensors, acoustic sensors, ultrasonic sensors, flow sensors, optical sensors, including cameras and/or infrared sensors, or combinations thereof. Sensor 104 detects respiration signals at sensor-user interface 112 in real time or intermittently. Either way, sensor 104 may enable respirator monitoring system 100 to track the respiration signals of user 102.

Sensor 104 converts the respiration signals into at least one sensor output signal. Sensor output signal may include any suitable signal, such as an electrical signal, an optical signal, or the like. Sensor 104 is communicatively coupled (e.g., connected) to computing device 106 via link 114. Link 114 includes any suitable wired connection (e.g., metal traces, fiber optics, Ethernet, or the like), a wireless connection (e.g., personal area network, local area network, metropolitan area network, wide area network, or the like), or a combination of both. For example, sensor 104 may include a communications unit that includes a network interface card, such as an Ethernet card, an optical transceiver, a radio frequency transceiver, a Bluetooth® interface card, WiFi′ radios, USB, or any other type of device that can send and receive information. In this way, sensor 104 generates an output representative of at least one respiratory parameter (e.g., data representative of a respiratory signal) that is received by computing device 106.

Computing device 106 includes any suitable computing device, such as a smartphone, a computerized wearable device (e.g., a watch, eyewear, ring, necklace, or the like), a tablet, a laptop, a desktop, or of the like. In some examples, computing device 106 and sensor 104 are two separate devices. In other examples, computing device 106 and sensor 104 are components of the same device. The output from sensor 104 is received by a processor of computing device 106. The processor of computing device 106 determines an occurrence of a breathing event based on the output. For example, the processor may communicate with one or more modules of computing device 106 to determine the occurrence of the breathing event such as detection of a breathing state based on the output. Computing device 106 may include a data storage to store sensor 104 output, the determined breathing event, or both (e.g., respiration data). By receiving sensor 104 output, determining breathing events based on the output, and storing respiration data, computing device 106 may enable respirator monitoring system 100 to track respiration data of user 102 over time.

Computing device 106 is communicatively coupled (e.g., connected) to user interface 108 via link 116. Link 116 may be the same or similar to link 114 discussed above. User interface 108 may include a graphical user interface (GUI), a display, a keyboard, a touchscreen, a speaker, a microphone, a gyroscope, an accelerometer, a vibration motor, or the like. In some examples, computing device 106 and user interface 108 are components of the same device, such as a mobile phone, a tablet, a laptop, or the like. In other examples, computing device 106 and user interface 108 are separate devices. Computing device 106 includes one or more output components that generate tactile output, audio output, video output, or the like that is received by user interface 108 to communicate information to user 102 or another entity. In this way, user interface 108 may notify user 120 of an occurrence of a breathing event. As one example, user interface 108 may receive an indication of the occurrence of a breathing event from computing device 106 and display on a display of user interface 108 information representative of the occurrence of the breathing event to user 102. Similarly, computing device 106 includes one or more input components that receive tactile input, kinetic input, audio input, optical input, or the like from user 102 or another entity via user interface 108. In this way, user interface 108 may receive user input from user 102 and send user input to computing device 106. For example, user 102 may provide user input to user interface 108, which communicates the user input to computing device 106. The user input (e.g., user data) includes, for example, information about age, gender, height, weight, and medical conditions; information associated with an occurrence of a breathing event, information associated with triggers generally or triggers of a respective occurrence of a breathing event, and the like. By communicatively coupling output components and input components of computing device 106 to user interface 108, user 102 (or another entity) may interact with computing device 106.

As shown in FIG. 1, sensor 104, computing device 106, and user interface 108 are optionally communicatively coupled to network 110. In other examples, fewer components (e.g., only computing device 106) may be coupled to network 110. Network 110 represents any public or private communication network, for instance, cellular, WiFi®, or other types of networks for transmitting data between computing systems, servers, and computing devices. Sensor 104, computing device 106, and user interface 108 may each be operatively coupled to network 110 using respective network links 118, 120, and 122. Network links 118, 120, and 122 may be any type of network connections, such as wired or wireless connections as discussed above. Network 110 may provide selected devices, such as sensor 104, computing device 106, and user interface 108 with access to the Internet, and may allow sensor 104, computing device 106, and user interface 108 to communicate with each other. For example, rather than communicating via link 114, sensor 104 and computing device 106 may communicate via network links 118 and 120. Similarly, rather than communicating via link 116, computing device 106 and user interface may communicate via network links 120 and 122. Additionally, sensor 104 may communicate directly with user interface 108 via network links 118 and 120.

In some examples, network 110 is operatively coupled to respiration monitoring platform 124, environmental information platform 126 and location information platform 128 using respective network links 130, 132, and 134. Network links 130, 132, and 134 may be the same or substantially similar to network links 118, 120, and 122 discussed above.

In some examples, user device 101 may send data to respiration monitoring platform 124, receive data from respiration monitoring platform 124, or both via network 110. For example, user device 101 may send respiratory data, user data, or both to respiration monitoring platform 124. In some examples, respiration monitoring platform 124 may store respiration data received from a plurality of users as captured by, for instance, sensors 104 and user data received from a plurality of computing devices (e.g., computing device 106). Respiration monitoring system 124 may analyze the respiration data and user data to determine breathing states representative of the plurality of users or to determine at least one of a breathing event, a trigger, and a trend related to the received respiration data, the received user data, or both. In this way, respiration monitoring system 124 may perform one or more functions discussed herein with respect to computing device 106. User device 101 also may receive data from respiration monitoring platform 124 including, for example, stored respiration data, stored user data, notification data (e.g., regarding a breathing event, a trigger, or a trend), algorithm data (e.g., to update or modify algorithms used by sensor 104 or computing device 106), and the like. In this way, respiratory monitoring system 100 may collect and analyze respiratory data and user data from at least one user to notify the at least one user of a breathing event, a trigger, or a trend relevant to the user.

In some examples, user device 101 may send data to environmental information platform 126, receive data from environmental information platform 126, or both via network 110. For example, computing device 106 may send location data (discussed below) to environmental information platform 126. Environmental information platform 126 may include a third-party application programmer interface (API) providing information related to local weather. Any suitable weather related API may be used, such as, for example, APIs available from The Weather Company, Atlanta, Ga. (e.g., “weather.com”) or Accuweather Inc., State College, Pa. In other examples, environmental information platform 126 may obtain some or all of the environmental information from other sources and aggregate the environmental information before forwarding the information to the computing device 106 of user in locations where the environmental information is relevant. In some example approaches, environmental information platform 126 may send to each user only the environmental information deemed relevant to the user.

Environmental information may include at least one environmental parameters such as, for example, temperature, barometric pressure, percent humidity, dew point, percent chance of precipitation, percent cloud cover, wind speed, air quality index (e.g., value, category, or both), dominant pollutants (e.g., the pollutants including but not limited to ozone, carbon monoxide, nitrous oxide, sulfur dioxide, ammonia, volatile organics, heavy metals, and particulate matter), pollen counts (e.g., relative ratings associated with different types of pollen, or the like), UV index, and the like. Environmental information may include current values, expected high values, expected low values, trend rising indicators, trend falling indicators, and the like for any one or more of the environmental parameters. In some examples, computing device 106 request from environmental information platform 126 real time (e.g., current) environmental information in response to determining a breathing event. Computing device 106 may receive the requested environmental information and associate the environmental information with the breathing event. In this way, respiration monitoring system 100 may obtain and associate real-time environmental information with real-time respiratory data. By associating real-time environmental data with real-time respiratory data, computing device 106 may determine a trigger of a breathing event based on the environmental data (e.g., an environmental trigger). Computing device 106 may store a plurality of environmental based triggers to determine a trend (e.g., an environmental trigger trend). Computing device 106 may notify user 102 of environmental information (e.g., current conditions) based on an environmental trigger or an environmental trigger trend. Such environmental information notifications enable user 102 to avoid environments that may cause hard breathing events.

In some examples, user device 101 may send data to location information platform 128, receive data from location information platform 128, or both via network 110. Location information platform 128 may include any suitable location service, such as the Global Positioning System (GPS), assisted GPS, WiFi® hotspot identification, cell tower identification, or the like. Additionally, location information platform 128 may include a map service. For example, computing device 106 includes software, hardware, or both to communicate with a respective location service, map service, or both. Computing device may send a request to location information platform 128 to identify the location of computing device 106 in response to determining an occurrence of a breathing event. Location information platform 128 may determine the location of computing device 106 (e.g., the event location) in response to an occurrence of a breathing event and send the event location to computing device 106. In this way, computing device 106 may associate an occurrence of a breathing event with an event location. Optionally, location information platform 128 may send at least a portion of a map showing the event location to computing device 106. In this way, computing device may communicate with user interface 108 to display an indication of an occurrence of a breathing event at the respective event location on a map.

In some examples, at least one of sensor 104, computing device 106, user interface 108, respiration monitoring platform 124, environmental information platform 126, and location information platform 128 may send data to or receive data from a third-party computing device (not shown) without traversing network 110. For example, sensor 104 may send respiratory data to a third-party computing device. The third-party computing device may send the respiratory data to respiration monitoring platform 124. The respiration monitoring platform 124 may analyze the respiratory data and send to computing device 106, via the third-party computing device, a breathing event, a trigger, or a trend based on the respiratory data. In this way, each component of respiration monitoring system 100 may communicate via a third-party device without the aid of network 110.

Respiratory monitoring system 100 may include one or more power sources (not shown). In some examples, one or more power source may be electrically coupled to each of sensor 104, computing device 106, and user interface 108. In other examples, one or more power sources may be electrically coupled to computing device 106, which may be electrically couple each of sensor 104, user interface 108, or both via links 114 and 116, respectively.

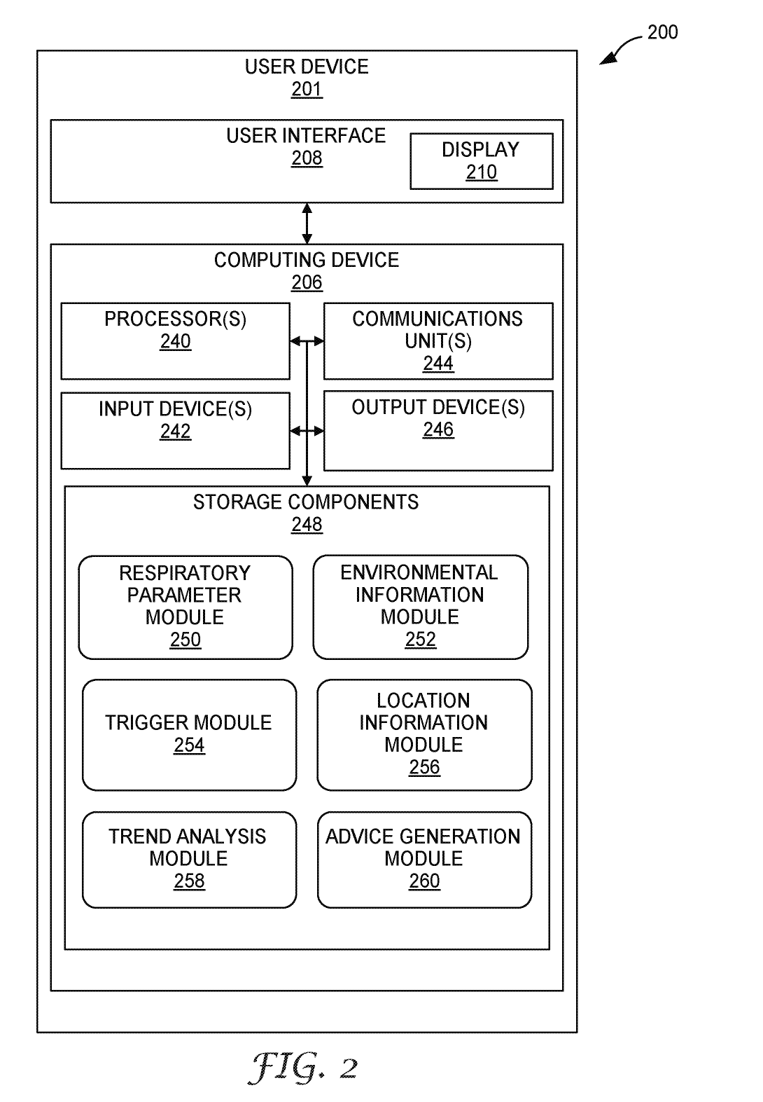

Although user device 101 of FIG. 1 includes sensor 104, computing device 106, and user interface 108, in some example approaches, user device 101 may include fewer or more components. FIG. 2 is a schematic and conceptual diagram illustrating an example user device 201 that includes a computing device 206 and a user interface 208. Computing device 206 and user interface 208 may be the same or substantially similar to computing device 106 and user interface 108, respectively, described above with respect to FIG. 1, except for the differences described herein. In one example approach, computing device 206 includes one or more processors 240, one or more one or more input devices 242, one or more communications units 244, one or more output devices 246, and one or more one or more storage components 248. In some examples, the one or more storage components 248 include respiratory parameter module 250, environmental information module 252, trigger module 254, location information module 256, trend analysis module 258, and advice generation module 260. In other examples, computing device 206 may include additional components or fewer components than those illustrated in FIG. 2.

One or more processors 240 are configured to implement functionality, process instructions, or both for execution within computing device 206. For example, processors 240 may be capable of processing instructions stored within one or more storage components 248. Examples of one or more processors 240 may include, any one or more of a microprocessor, a controller, a digital signal processor (DSP), an application specific integrated circuit (ASIC), a field-programmable gate array (FPGA), or equivalent discrete or integrated logic circuitry.

Computing device 206 also includes one or more input devices 242. Input devices 242, in some examples, are configured to receive input from a user through tactile, audio, or video sources. Examples of input devices 242 include a mouse, a button, a keyboard, a voice responsive system, video camera, microphone, touchscreen, or any other type of device for detecting a command from a user. In some example approaches, user interface 208 includes all input devices 242 employed by computing device 206.

Computing device 206 further includes one or more communications units 244. Computing device 206 may utilize communications units 244 to communicate with external devices (e.g., sensor 104, user interface 108 or 208, respiratory parameter platform 124, environmental information platform 128, and/or location information platform 128) via one or more networks, such as one or more wired or wireless networks. Communications units 244 may include a network interface card, such as an Ethernet card, an optical transceiver, a radio frequency transceiver, or any other type of device that can send and receive information. Communications units 244 may also include WiFi′ radios or a Universal Serial Bus (USB) interface. In some examples, computing device 206 utilizes communications units 244 to wirelessly communicate with an external device such as a server.

Computing device 206 may further include one or more output devices 246. Output devices 246, in some examples, are configured to provide output to a user using, for example, audio, video or tactile media. For example, output devices 246 may include display 210 of user interface 208, a sound card, a video graphics adapter card, or any other type of device for converting a signal into an appropriate form understandable to humans or machines. In some example approaches, user interface 208 includes all output devices 246 employed by computing device 206.

One or more storage components 248 may be configured to store information within computing device 206 during operation. One or more storage components 248, in some examples, include a computer-readable storage medium or computer-readable storage device. In some examples, one or more storage components 248 include a temporary memory, meaning that a primary purpose of one or more storage components 248 is not long-term storage. One or more storage components 248, in some examples, include a volatile memory, meaning that one or more storage components 248 does not maintain stored contents when power is not provided to one or more storage components 248. Examples of volatile memories include random access memories (RAM), dynamic random-access memories (DRAM), static random-access memories (SRAM), and other forms of volatile memories known in the art. In some examples, one or more storage components 248 are used to store program instructions for execution by processors 240. One or more storage components 248, in some examples, are used by software or applications running on computing device 206 to temporarily store information during program execution.

In some examples, one or more storage components 248 may further include one or more storage components 248 configured for longer-term storage of information. In some examples, one or more storage components 248 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

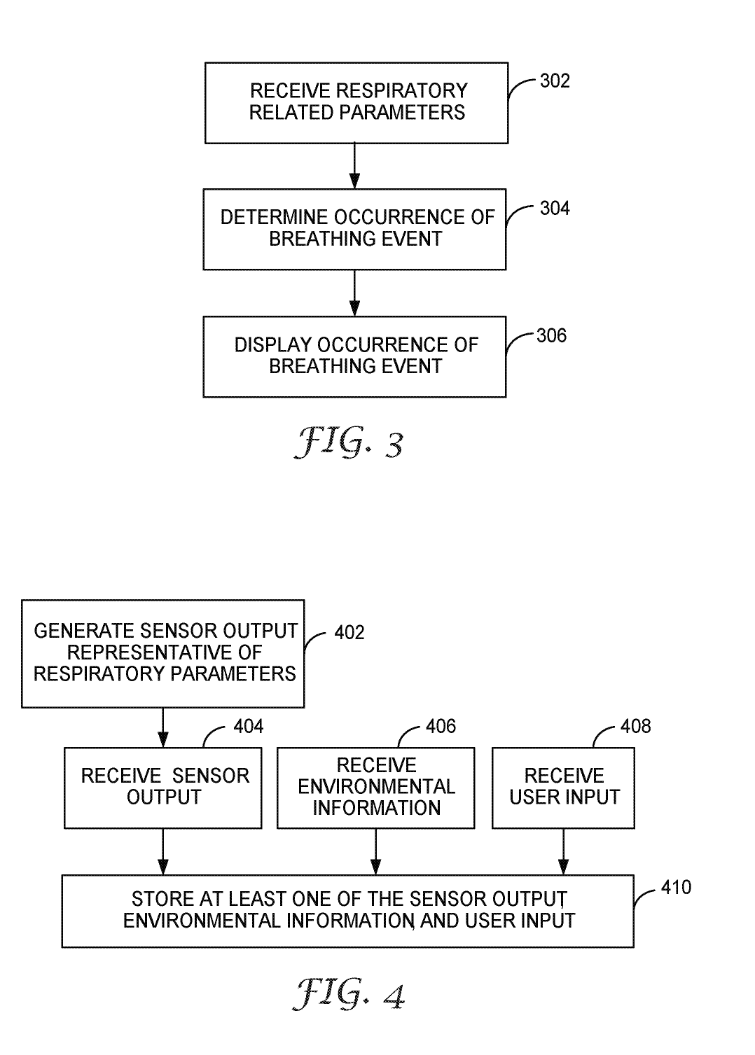

As noted above, computing device 206 also may include respiratory parameter module 250, environmental information module 252, trigger module 254, location information module 256, trend analysis module 258, and advice generation module 260. Each of respiratory parameter module 250, environmental information module 252, trigger module 254, location information module 256, trend analysis module 258, and advice generation module 260 may be implemented in various ways. For example, one or more of respiratory parameter module 250, environmental information module 252, trigger module 254, location information module 256, trend analysis module 258, and advice generation module 260 may be implemented as an application or a part of an application executed by one or more processors 240. In other examples, one or more of respiratory parameter module 250, environmental information module 252, trigger module 254, location information module 256, trend analysis module 258, and advice generation module 260 may be implemented as part of a hardware unit of computing device 206 (e.g., as circuitry). In other examples, one or more of respiratory parameter module 250, environmental information module 252, trigger module 254, location information module 256, trend analysis module 258, and advice generation module 260 may be implemented remotely on a third-party device as part of an application executed by one or more processors of the third-party device or as a hardware unit of the third-party device (e.g., respiration parameter platform 124, environmental platform 126, and location information platform 128). Functions performed by one or more of respiratory parameter module 250, environmental information module 252, trigger module 254, location information module 256, trend analysis module 258, and advice generation module 260 are explained below with reference to the example flow diagrams illustrated in FIGS. 3-8.

In one example approach, signals from a wearable respiratory sensor 104 are received by computing device 106 or by computing device 206 and forwarded to respiratory monitoring platform 124 for analysis. In one such example approach, respiratory monitoring platform 124 receives respiratory parameter signals from one or more computing devices 106 or 206, associates each signal with a user and determines, from each signal, characteristics of a breathing pattern associated with a breathing disorder such as asthma. In one example approach, sensor 104 measures the pressure exerted by a user's chest or abdomen against a piece of clothing, such as a belt or elastic band. In one such example approach, signal processing algorithms identify characteristics or features in a repeated breath pattern, such as, for example, inhalation time, exhalation time, relative tidal volume and relative air flow rate, as detected by, for instance, sensor 104. By comparing these characteristics to a dictionary of respiratory features derived from both normal and asthmatic people, respiratory monitoring platform 124 may calculate, for each user, a breathing score which correlates to the severity of their asthma symptoms. In addition, respiratory monitoring platform 124 may calculate, for each user, parameters associated with two or more breathing states identified for the user and may, in some instances, modify the parameters based on user input to tune breathing state detection to the individual user.

Computing device 206 may also include additional components that, for clarity, are not shown in FIG. 2. For example, computing device 206 may include a power supply to provide power to the components of computing device 206. Similarly, the components of computing device 206 shown in FIG. 2 may not be necessary in every example of computing device 206.

FIG. 3 is a flow diagram of an example technique for notifying a user of an occurrence of a breathing event. Although the technique of FIG. 3 will be described with respect to respiratory monitoring system 100 of FIG. 1 and computing device 206 of FIG. 2, in other examples, the technique of FIG. 3 may be performed using a different system, a different computing device, or both. Additionally, respiratory monitoring system 100 and computing device 206 may perform other techniques for notifying a user of an occurrence of a breathing event.

The technique illustrated in FIG. 3 includes receiving, by computing device 206, for example, respiration parameter module 250, at least one output representative of at least one respiratory related parameter (302). Respiratory related parameters include respiratory parameters, environmental information, location information, user information, and the like. In some examples, respiratory parameters include amplitudes, rates, and durations of inhalation, exhalation, or both. In other examples, respiratory parameters may include other parameters indicative of respiration, such as ventilation flow rate, tidal volumes, consistency of respiration rate, pulse rate, oxygen-saturation, acoustic spectral density, and the like. In some examples, sensor 104 generates the at least one output representative of the at least one respiratory related parameter. In other examples, computing device 206 receives the at least one output representative of the at least one respiratory related parameter from other sources, such as, for example, user interface 208, respiration monitoring platform 124, environmental information platform 126, location information platform 128, a third-party computing device, or the like.

After receiving the at least one output representative of the at least one respiratory related parameter, the technique illustrated in FIG. 3 includes determining, by computing device 206, for example, respiration parameter module 250, an occurrence of a breathing event based on the at least one output (304). As discussed above, a breathing event may include detecting a smooth breathing state (e.g., improved breathing relative to average ventilation), detecting a hard breathing state (e.g., worse breathing relative to average ventilation), or the like. In some examples, determining the occurrence of the breathing event based on the at least one output includes comparing the at least one output representative of the at least one respiratory related parameter to a respective baseline respiratory related parameter. For example, where the output representative of the at least one respiratory related parameter includes an amplitude and frequency (e.g., an average rate of a repeated respiratory parameter) of respiration of user 102, the respective baseline respiratory related parameter may include an average amplitude and frequency of respiration of user 102. Additionally, or alternatively, the respective baseline respiratory related parameter may include an average amplitude and frequency of respiration of an average person with similar or substantially the same physical characteristics as user 102 (e.g., age, gender, height, weight, medical conditions, or the like). In other examples, other respiratory related parameters, such as duration of inhalation or amplitude, frequency, or duration of exhalation, may be compared to a respective baseline respiratory related parameter.

In other examples, one or more different approaches may be used to determine a baseline respiratory related parameter. In one example approach, user 102 may rest for a specified time interval (e.g., 5 minutes), during which sensor 104 measures and determines a set of average baseline respiratory parameters (e.g., normal breathing. In another example approach, computing device 106 selects a time period near the beginning of each day and gathers information from sensor 104 to establish baseline respiratory parameters for the respective day. In another example approach, computing device 106 refers to a respiratory parameter module 250 which stores a histogram of respiratory parameters collected over a period of several days to several weeks. In this approach, a baseline respiratory parameter may be defined as any parameter that falls within one standard deviation of the average over several days or weeks. In another example approach, the computing device 106 gathers statistical information from the respiratory monitoring platform 124. In this approach, baseline respiratory parameters may be based on one or more characteristics of one or more sub-groups of users. For instance, one can define a baseline as a respiratory parameter which is typical of patients within a certain nursing home, or which is typical for users who have been prescribed on a new respiratory medication for more than two weeks, or which is typical for residents of a city when the weather conditions exceed certain limiting values of, for instance, temperature or humidity or air quality levels, or the like.

In some examples, the baseline respiratory related parameters may be stored by computing device 206, for example, respiration parameter module 250. In other examples, the baseline respiratory related parameters may be stored by respiration monitoring platform 124, a third-party computing device, or the like. The baseline respiratory related parameters may be initially determined based on user input. For example, initial baseline respiratory related parameters may be based on preexisting baseline respiratory related parameters determined for person having a similar age, gender, height, weight, medical conditions, or the like compared to user 102. In this way, computing device 206 may be configured to determine baseline respiratory parameters that are personalized to user 102.

In some examples, the baseline respiratory parameters are fixed. In other examples, the baseline respiratory parameters vary over time. For example, computing device 206 may adjust (e.g., modify or update) one or more baseline respiratory parameters at regular or irregular intervals, in response to user input, determined occurrences of breathing events, data received from respiration monitoring platform 124, or the like. By adjusting the baseline respiratory parameters, computing device 206 may improve the accuracy of the baseline respiratory parameters, assist user 102 in changing the average respiration of user 102 over time, or the like.

In some examples, comparing the at least one output representative of the at least one respiratory related parameter to a respective baseline respiratory related parameter includes determining whether the at least one output representative of the at least one respiratory related parameter is within a respective threshold value of the respective baseline respiratory related parameter. In some examples, the respective threshold values may be fixed. For example, the respective threshold values may be predetermined based on user input. In other examples, the respective threshold values may vary over time. For example, in response to user input, computing device 206 may adjust (e.g., modify or update) one or more baseline respiratory parameters at regular or irregular intervals, in response to user input, determined occurrences of breathing events, data received from respiration monitoring platform 124, or the like. By adjusting the baseline respiratory parameters, computing device 206 may improve the accuracy of the comparison of the at least one output representative of the at least one respiratory related parameter to a respective baseline respiratory related parameter. In other examples, the determination of a breathing event may depend on contextual information. For example, the threshold value used to determine a breathing event may be adjusted according to one or more of a location of user 102 (e.g., whether user 102 is indoors or outdoors), environmental information (e.g., whether the user has been exposed to unusual environmental factors, such as extreme weather or air quality issues), activity of user 102 (e.g., whether the user is physically active or at rest), and breath event history (e.g., whether the user has reported other respiratory symptoms within the past 24 hours).

After determining the occurrence of a breathing event, the technique illustrated in FIG. 3 includes displaying, on display 210 of user interface 208, an indication of the occurrence of the breathing event (306). For example, as discussed above, computing device 206 includes output devices 246. Output devices 246 provide video output via user interface 208 to display 210. Additionally, or alternatively, output devices 246 may provide other output media to a user, such as tactile output, audio output, or the like. By providing output media to user interface 208, computing device 206 may notify user 102 of an occurrence of a breathing event. In other examples, computing device 206 may utilize communications units 244 to communicate the indication of the occurrence of the breathing event with one or more external devices (e.g., respiration monitoring platform 124, one or more third-party computing devices, or the like) via network 110. By communicating the indication of the occurrence of the breathing event with one or more external devices, computing device 206 may notify user 102 of an occurrence of a breathing event by the one or more external devices.

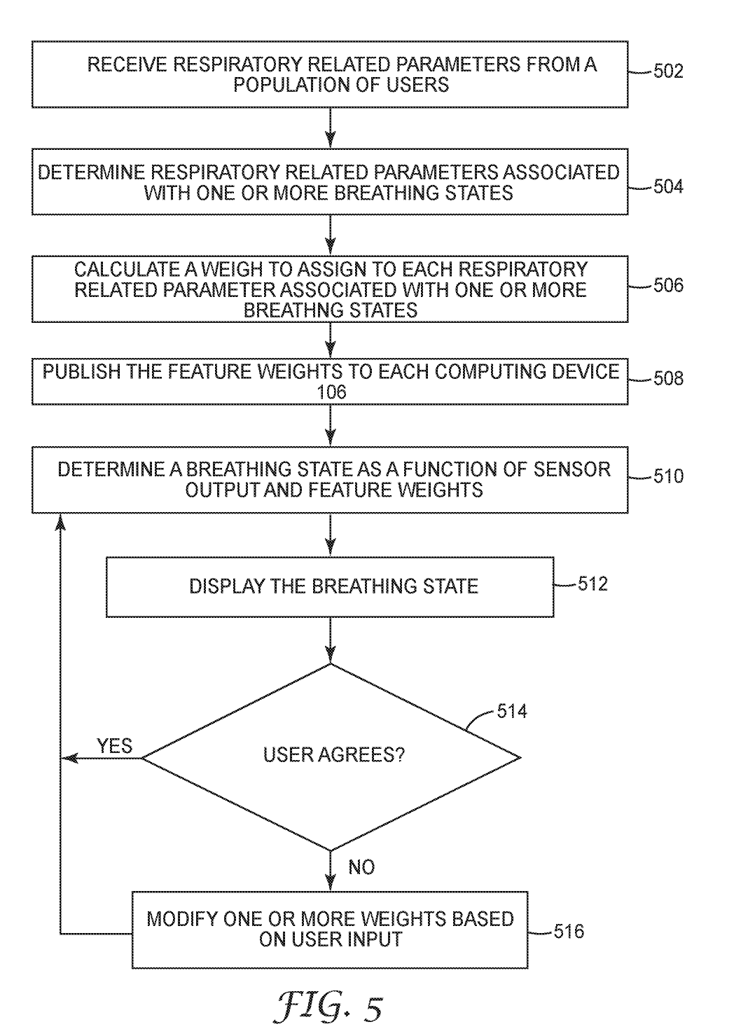

In some examples, receiving respiratory related parameters (302) includes receiving at least one of sensor output, environmental information, and user input. FIG. 4 is a flow diagram of an example technique for receiving, by computing device 206, respiratory related parameters. Although the technique of FIG. 4 will be described with respect to system 100 of FIG. 1 and computing device 206 of FIG. 2, in other examples, the technique of FIG. 4 may be performed using a different system, a different computing device, or both. Additionally, system 100 and computing device 206 may perform other techniques for receiving respiratory related parameters.

The technique illustrated in FIG. 4 includes generating, by sensor 104, at least one output representative of at least one respiratory parameter (402). For example, as discussed above with respect to FIG. 1, sensor 104 includes at least one sensor that detects at least one signal indicative of respiration characteristics of user 102 (e.g., respiration signals) at a sensor-user interface 112. Sensor 104 converts the respiration signals into at least one sensor output signal, such as an electrical signal, an optical signal, or the like. In this way, sensor 104 generates at least one output representative of at least one respiratory parameter.

After generating at least one output representative of at least one respiratory parameter (402), the technique illustrated in FIG. 4 includes receiving, by computing device 206, the at least one output representative of the at least one respiratory parameter (404). For example, as discussed above with respect to FIG. 1, sensor 104 is connected to computing device 106 (e.g., computing device 206) via link 114. Computing device 206 includes one or more communications units 244 to communicate with external devices (e.g., sensor 104). In this way, computing device 206 receives the at least one output representative of at least one respiratory parameter.

Optionally, the technique illustrated in FIG. 4 includes receiving, by computing device 206, environmental information (406). For example, as discussed above with respect to FIG. 1, environmental information may include environmental parameters obtained from a third-party API of environmental information platform 126. Environmental information platform 126 is connected to computing device 106 (e.g., computing device 206) via network link 132. Computing device 206 includes one or more communications units 244 to communicate with external devices (e.g., environmental information platform 126). In this way, computing device 206 receives environmental information from environmental information platform 126. In some example approaches, environmental information also includes location information provided by computing device 106, or received from external devices (e.g., location information platform 128), or both. For example, as discussed above with respect to FIG. 1, representative location information may include location data derived from a GPS signal, map data, or both.

Optionally, the technique illustrated in FIG. 4 includes receiving, by computing device 206, user input (408). For example, as discussed above with respect to FIG. 1, user interface 108 (e.g., user interface 208) may receive user input from user 102 and send user input to computing device 106 (e.g., computing device 206). The user input (e.g., user data) includes, for example, information about age, gender, height, weight, and medical conditions; information associated with an occurrence of a breathing event, information associated with triggers generally or triggers of a respective occurrence of a breathing event, and the like. In this way, computing device 206 receives user input from user 102 via user interface 208.

After receiving sensor output and, optionally, environmental information and user input, the technique illustrated in FIG. 4 includes storing, by computing device 206, for example, one or more of storage components 248, at least one of the sensor output, the environmental information, and the user input (410). By storing at least one of the sensor output, the environmental information, and the user input, computing device 106 may associate an occurrence of a breathing event with one or more of the sensor output, the environmental information, the user input, and the location information.

In some examples, machine learning is used to determine relevant respiratory related parameters and to calculate weights to be associated with such respiratory related parameters when determining the occurrence of a breathing event, or the detection of a breathing state, trigger or trend. In some such examples, respiratory monitoring platform 124 receives potential respiratory related factors collected from a plurality of users. Some of the users exhibit what is classified as normal breathing, while others exhibit breathing patterns associated with certain breathing disorders. By comparing the respiratory related parameter information to a dictionary of respiratory related parameters, derived, for instance, from both normal and asthmatic people, respiratory monitoring platform 124 may calculate, for each user, a breathing score which correlates to the severity of their asthma symptoms. In addition, respiratory monitoring platform 124 may determine, for each user, respiratory related parameters most relevant to determining their breathing state.

Lightweight and wearable physiological monitors may monitor and record a variety of health and wellness conditions. Such monitors may be used by, for instance, respiratory monitoring platform 124 to detect parameters relevant to determining a respiratory function of a user. For instance, respiratory monitoring platform 124 may be used to detect the severity of and to monitor breathing disorders such as asthma and chronic obstructive pulmonary disease (COPD). Monitoring methods may include detection of chemical elements from exhaled breath, acoustic sounds (cough or wheeze) detected at the chest or throat, and measurements of the relative motion in the chest wall or abdomen. In one example approach, sensor 104 includes a device that measures respiration based on the pressure exerted between the chest or abdomen and a piece of clothing, such as a belt or elastic band, as described in more detail below. In one such example approach, the device measures respiratory related parameters such as the cycle time of a respiratory cycle, and one or more of the tidal volume, inspiratory flow, inspiratory pause, expiratory flow and expiratory pause for each person being measured.

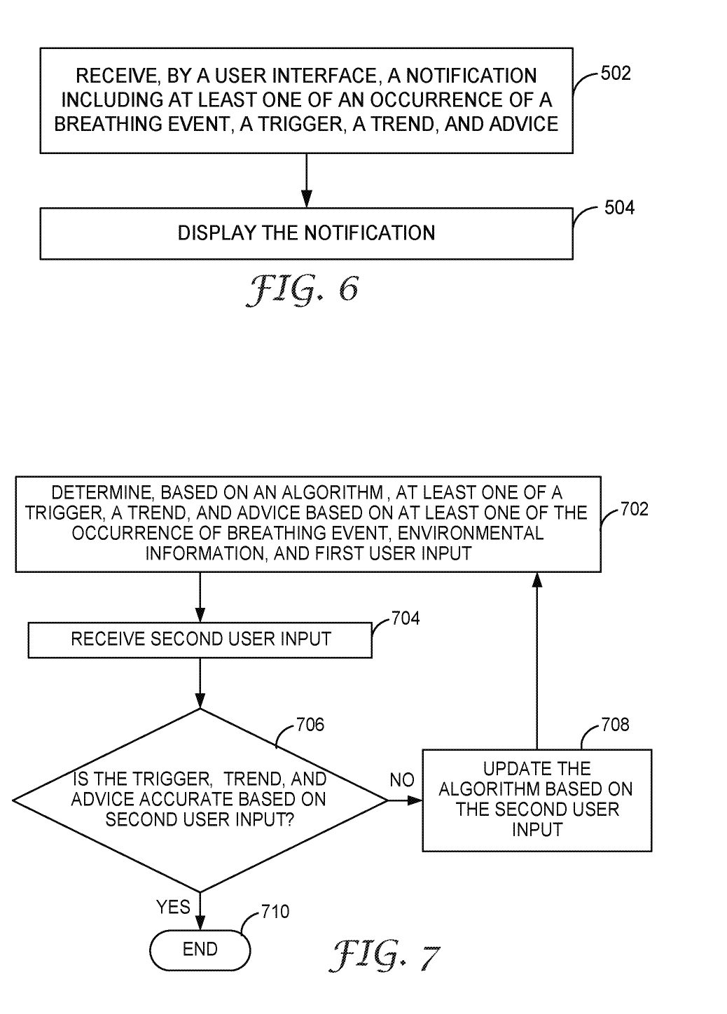

FIG. 5 is a flow diagram of an example technique for detecting a condition such as a breathing state of a user. Although the technique of FIG. 5 will be described with respect to system 100 of FIG. 1 and computing device 206 of FIG. 2, in other examples, the technique of FIG. 5 may be performed using a different system, a different computing device, or both. Additionally, system 100 and computing device 206 may perform other techniques for receiving respiratory related parameters.

The technique illustrated in FIG. 5 includes receiving potential respiratory related parameters collected from a population of users (502). In an example based on the detection and monitoring of asthma, the population of users includes people with and without asthma. The asthma condition is characterized by physiologists as an airway obstruction, usually caused by bronchial inflammation. Asthma is described as a reversible airway obstruction in that certain drugs, such as the beta agonist albuterol, can partially reverse the condition and allow for temporary relief in the asthma subject.

In one example approach, respiratory monitoring platform 124 determines respiratory related parameters that help distinguish between an asthmatic breathing state and a normal breathing state, and that help distinguish asthmatic breathing states from other common conditions such as rapid breathing due to physical exercise or excitement (504). In one example approach, respiratory monitoring platform 124 seeks to detect signs of airway obstruction. Respiratory monitoring platform 124 then assigns weights to the respiratory related parameters deemed most relevant in determining a given condition (such as a breathing state, breathing event, trigger or trend) (506) and stores an indication of the relevant parameters in a feature vector. Respiratory monitoring platform 124 then publishes the weights associated with each feature in the feature vector for that condition to computing devices 106 (508).

In one example approach, computing device 106 receives the weights associated with each feature in a feature vector and applies the weights to respiration related parameters received at the computing device 106 to determine if a condition has been met (510). For instance, a feature vector associated with detection of labored asthmatic breathing may include features associated with respiration related parameters received from sensor 104 and features associated with respiration related parameters receive as environmental information. As one example, a feature vector is a string of coded words. Each word in the string represents a respiratory related parameter, a weight assigned to a respiratory parameter, or the like. In this way, the feature vector provides an efficient approach to summarize, store in memory, or both all, or nearly all, of the respiratory related parameters used by computing device 106 (or respiratory monitoring platform 124) to detect, determine, or evaluate a breathing state.

In one example approach, when a condition is met, the user is notified via user interface 108 (512). In one such example approach, the user may indicate whether he or she agrees with the determination (514). If the user agrees with the determination (YES branch), computing device 106 continues to monitor respiration related parameters at 510.

If, however, the user does not agree with the determination (NO branch), computing device 106 modifies one or more weights of features in the feature vector to reflect the feedback received from the user (516) before returning to monitor respiration related parameters at 510.

In one example approach, as noted above, respiratory monitoring platform 124 determines both the feature vector and the weights used, at least initially, by computing devices 106. For example, respiratory monitoring platform 124 may classify the severity of asthma from breathing patterns recorded from a pressure sensor on a user's abdomen. In one example approach, respiratory monitoring platform 124 applies a preprocessing algorithm to reject breathing patterns with low Signal-to-Noise-Ratio (SNR) and then applies a feature extraction method to develop a feature vector for each condition based on relevant respiration related parameters. Respiratory monitoring platform 124 then determines, for instance, whether the breathing features collected across a population of users may be used, for example, to classify the severity of the asthma symptoms of each patient.

A representative preprocessing algorithm is discussed next. Since, in the example above, the breathing signal coming from a pressure sensor is a continuous signal, in one example approach, different instances of breathing are first segmented and synchronized with each other. This may involve, for instance, detecting a beginning time-point and the ending time-point of each breath.

The time duration of each breath may then be calculated and stored as one of the features representing the breath signal. In one example approach, when synchronizing the breathing signals, the duration time of each breathing signal is scaled such that each time-scaled breathing signal lasts the same amount of time. To keep the same number of samples in the breathing signals, in one example approach, the time-scaled signals are resampled to have same number of samples.

In one example approach, the average of the breathing signals may be subtracted from the breathing signal to zero-mean the breathing signals. In addition, in some example approaches, respiratory monitoring platform 124 applies a threshold for minimum breathing volume to discard breathing signals where the sum of the absolute value of the zero-mean breathing signal is lower than the chosen threshold. Such breathing signals are considered as low SNR signals and may be ignored. After the low SNR signals are removed, in one example approach, the remaining breathing signals are normalized to have the same breathing volume (which can be represented by the sum of the absolute value of the breathing signal value).

A method of feature extraction will be discussed next. In one example approach, respiratory monitoring platform 124 uses respiration related parameters such as physiological parameters, environmental parameters, user information and measured sensor 104 data received from the preprocessing step above to train one or more classification algorithms. In one such example approach, respiratory monitoring platform 124 uses a labeled set of breathing features received from a population of subjects to train each classification algorithm.

For instance, in one example approach, respiratory monitoring platform 124 uses a labeled set of aggregated breathing features to train a classification algorithm to predict the severity of asthma symptoms. In one such approach, respiratory monitoring platform 124 receives a labeled set of breathing features gathered over time. For instance, such a labeled training set may be generated by presenting a questionnaire about severity of asthma symptoms to the subjects of an experimental study, every 8 hours, while monitoring each subject's breathing. The questionnaire responses are then transformed to represent asthma symptoms severity as a scalar value. Respiratory monitoring platform 124 then applies a classification algorithm such as logistic regression or decision tree to estimate the likelihood that the respiratory related parameters associated with the features in the feature vector can be used to distinguish severe asthma symptoms from healthy or normal breathing. If successful, respiratory monitoring platform 124 may isolate a scalar value representative of asthma severity, or “asthma severity score,” and be able to use this asthma severity score as feedback to the wearer of the sensor 104.

In another example approach, the respiratory parameter module 250 stores a generalized feature vector, with un-weighted respiratory related parameters pending refinement based upon user input. In one such approach the respiratory parameter module 250 refines the feature vector based upon a labeled set of breathing features gathered over time. For instance, such a labeled training set may be generated by presenting a questionnaire about severity of asthma symptoms to each new user, every 8 hours, while monitoring the user's breathing. The questionnaire responses are then transformed to represent asthma symptoms severity as a scalar value. Respiratory parameter module 250 then applies a classification algorithm such as logistic regression or decision tree to estimate the likelihood that the respiratory related parameters associated with the features in the feature vector can be used to distinguish severe asthma symptoms from healthy or normal breathing.

In one some example approach, users 102 monitor variations in the asthma severity scores generated by computing device 106 over a period of several days to several weeks. Such an approach may then be used by the user to track variations in environmental influences or in the management of their disease, with a sensitivity and consistency difficult to derive from their own self-diagnosis.

In one example approach, respiratory monitoring platform 124 discards from the aggregated feature vector, features that have minimal impact on the asthma severity score, or whose impact falls below a selected threshold. Respiratory monitoring platform 124 then assigns weights representing the contribution of each feature in the aggregated feature vector to the severity score and distributes the aggregated feature vector and its associated weights to each computing device 106.

In one example approach, respiratory monitoring platform 124 uses a quantization method similar to Voronoi-tessellation to reduce the dimensionality of the feature vector. In this method, a k-means clustering algorithm with “L” number of clusters assigns a cluster index to each feature vector. Each feature vector may, therefore, be represented as an integer scalar number (with a value between 1 and L).

Returning to the example of the asthma severity score discussed above, since there is a natural variation in breathing signal instances, a decision regarding the severity of asthma symptoms should be made based on observed variation in breathing signal patterns during a long period of time (i.e., over an hour or several hours). To represent the variation in the measured breathing signals in this period of time, in one example approach, respiratory monitoring platform 124 employs a histogram of the quantized breathing patterns to represent the change in patterns over time. Such a histogram may represent, for example, the frequency of the occurrence of different breathing patterns during that period of time and may be represented as an L-dimensional vector termed an aggregated feature vector. Other lossy and lossless compression techniques may also be used to collect the requisite respiration related parameters over time. In such an approach, respiratory monitoring platform 124 uses a labeled set of aggregated breathing features to train a classification algorithm to predict the severity of asthma symptoms. As above, respiratory monitoring platform 124 may receive a labeled set of aggregated breathing features gathered over time. Respiratory monitoring platform 124 then applies a classification algorithm such as logistic regression or decision tree to estimate the likelihood that the aggregated feature vector represents severe asthma symptoms versus a healthy or normal breathing. If successful, respiratory monitoring platform 124 may isolate a scalar value representative of the asthma severity score, and be able to use this asthma severity score as feedback to the wearer of the sensor 104.

In some examples, one or more functions of respiratory monitoring platform 124, as discussed above with respect to FIG. 5, may be performed by computing device 106. For example, computing device 106 may receive from respiratory monitoring platform 124 an un-weighted feature vector. After receiving the un-weighted feature vector, computing device 106 may receiving user input during a training period (e.g., computing device 106 may gather information from survey questions from the user over one or more days). After receiving user input, computing device 106 may determine (e.g., calculate) an initial set of weights for use in the detection algorithm. After determining an initial set of weights, computing device 106 may receive subsequent user input and incrementally adjust the weights based on the subsequent user input. In this way, computing device 106 may be configured to adjust the classification algorithm.

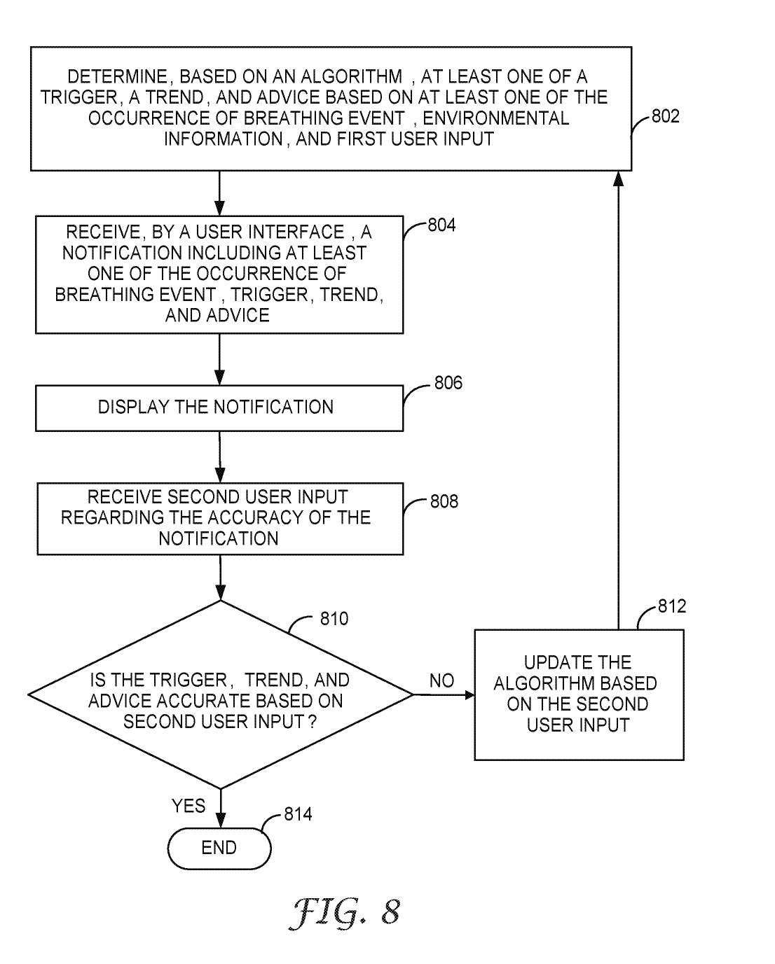

In some examples, displaying the occurrence of the breathing event (306) includes communicating a notification to user 102 via a display of the user interface. FIG. 6 is a flow diagram of an example technique for displaying a notification to user 102 via display 210 of user interface 208. Although the technique of FIG. 6 will be described with respect to system 100 of FIG. 1 and computing device 206 of FIG. 2, in other examples, the technique of FIG. 6 may be performed using a different system, a different computing device, or both. Additionally, system 100 and computing device 206 may perform other techniques for displaying a notification to a user.

The technique illustrated in FIG. 6 includes receiving, by user interface 208, a notification (602). For example, as discussed above with respect to FIG. 3, computing device 206 includes output devices 246. Output device 246 may provide video output to display 210 via user interface 208. Additionally, or alternatively, output device 246 may provide other media output (e.g., tactile output, audio output, or the like) to user interface 208 or other external devices. The notification includes an indication of at least one of a breathing state, an occurrence of a breathing event, a trigger associated with at least one occurrence of a breathing event, a trend associated with at least one occurrence of a breathing or at least one trigger, and advice associate with a breathing state, at least one occurrence of a breathing event, at least one trigger, at least one trend, or the like. In this way, the technique illustrated in FIG. 6 includes receiving, by user interface 208, a notification.

After receiving the notification, the technique of FIG. 6 includes displaying, by display 201, the notification (604). For example, display 210 of user interface 208 may transform the video output into one or more images that are understandable to humans or machines. Additionally, or alternatively, user interface 208 (or, in some examples, other external devices) may transform other media output (e.g., tactile output, audio output, or the like) into one or more of a movement, physical feature, sound, or the like that are understandable to humans or machines. By transforming the notification received by user interface 208 into a media that is understandable by a human or machine, respiratory monitoring system 100 may communicate the notification to a human (e.g., user 102) or a machine.

In some examples, computing device 206 queries location information module 256 to determine location information that is temporally associated with, for example, the occurrence of a breathing event before transmitting such information to a user via display 210 of user interface 208. For example, computing device 206 may direct processor 240 to store in location information module 256 a present location of user 102. Location information module 256 may then communicate the location information to at least one of storage components 248 (e.g., at least one of respiratory parameter module 250, environmental information module 252, trigger module 254, trend analysis module 258, and advice generation module 260).

In some example approaches, computing device 206 may determine whether a breathing event has occurred based on a location that is associated with sedentary activity of user 102, such as an abode or place of work of user 102, or dynamic activity of user 102, such as a gym or when user 102 is moving. As another example, computing device 206 may determine whether a breathing event has occurred based on a location that is associated with one or more occurrences of breathing events, such as a location having an allergen to which user 102 is sensitive. By basing the algorithm on location information, respiratory monitoring system 100 may more accurately determine at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

In some examples, environmental information module 252 uses the location information to determine, by environmental information platform 126, one or more environmental parameters temporally associated, spatially associated, or both with the occurrence of the breathing event. For example, in response to determining an occurrence of a breathing event, computing device 206 causes environmental information module 252 to determine from environmental information platform 152 one or more environmental parameters based on the present location of user 102. In other examples, environmental information module 252 may determine one or more environmental parameters based on user input, estimated spatial information associated with user 102 (e.g., a last known location of user 102, the location of an abode or place of work of user 102, or the like), or the like. Environmental information module 252 may communicate one or more environmental parameters to at least one of storage components 248 (e.g., at least one of respiratory parameter module 250, trigger module 254, trend analysis module 258, and advice generation module 260).

Computing device 206 may determine whether a breathing event has occurred based on one or more environmental parameters. For example, computing device 206 may determine the algorithm based on one or more of temperature, barometric pressure, percent humidity, dew point, percent chance of precipitation, percent cloud cover, wind speed, air quality index (e.g., value, category, or both), dominant pollutants, pollen counts (e.g., relative ratings associated with different types of pollen, or the like), UV index, like environmental parameters, or expected values of such environmental parameters or trends of values of such environmental parameters. As one example, detection may be based on an air quality index of a present or predicted location of user 102, such that computing device 206 generates a notification to user 102 indicating a probability of an occurrence of a breathing event. By basing detection of a breathing event on one or more environmental parameters, respiratory monitoring system 100 may more accurately determine at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

In some examples, trigger module 254 uses at least one of an occurrence of a breath event (e.g., obtained from respiratory parameter module 250), a location of user 102 (e.g., obtained from location information module 256), and one or more environmental parameters (e.g., obtained from environmental information module 252) to determine a trigger associated with a respective occurrence of a breathing event. A trigger is associated with an occurrence of a breathing event when the trigger is related to the occurrence of one or more breathing events (e.g., temporal concurrence or spatial correlation), causes the occurrence of one or more breathing events (e.g., a contributing factor, an originating factor, or an exclusive factor), or the like. In some examples, a trigger includes a potential trigger. A potential trigger may be based on at least one of location information, one or more environmental parameters, or user input before the occurrence of a breathing event. Trigger module 250 may communicate the occurrence of the breathing event to at least one of storage components 248 (e.g., at least one of respiratory parameter module 250, environmental information module 252, trigger module 254, location information module 256, trend analysis module 258, and advice generation module 260). By determining and communicating triggers, respiratory monitoring system 100 can notify user 102 of triggers.

In some examples, a determination that a particular location is associated with an occurrence of a breathing event may be based on two or more breathing events occurring in the particular location, on user input (e.g., the user identifies a location as more likely than not to be associated with an occurrence of a breathing event), or the like. As one example, trigger module 254 may determine that a particular outdoor area is a trigger when the particular outdoor area is associated with two or more hard breathing events.

In the same or different examples, a determination that one or more environmental parameters are associated with an occurrence of a breathing event may be based on two or more breathing events occurring when the one or more environmental parameters occur in proximity to user 102. For example, trigger module 254 may determine that a particular air quality index is a trigger when two or more breathing events occur in a location having an air quality index equal to or greater than a particular air quality index value (air quality indices range from 0, most good, to 500, most hazardous).

In some example approaches, computing device 206 may notify a user 102 based on one or more triggers. As one example, notification may be based on a trigger associated with a particular location or one or more environmental parameters, such that the notification is based on a probability of an occurrence of a breathing event in the particular location, when the one or more environmental parameters are present, or both. By basing the notification on one or more triggers, respiratory monitoring system 100 may more accurately determine at least one of an occurrence of a breathing event, a second trigger associated with at least one occurrence of a breathing event, a trend associated with at least one occurrence of a breathing or at least one trigger, advice associate with at least one occurrence of a breathing event, at least one trigger, at least one trend, or the like.

In some examples, trend analysis module 258 uses two or more occurrences of a breathing event (e.g., obtained from respiratory parameter module 250), a location of user 102 (e.g., obtained from location information module 256), one or more environmental parameters (e.g., obtained from environmental information module 252), and one or more triggers (e.g., obtained from trigger module 254) to determine a trend associated with a respective occurrence of a breathing event. A trend identifies a plurality of occurrences of breathing events that are associated with one or more locations, one or more environmental parameters, user input, or the like, as discussed above. For example, trend analysis module 258 may determine a notification that indicates one or more locations, one or more environmental parameters, user input, or the like that are associated with a plurality of occurrences of breathing events. Trend analysis module 250 may communicate a trend to at least one of storage components 248 (e.g., at least one of respiratory parameter module 250, environmental information module 252, trigger module 254, location information module 256, and advice generation module 260). By determining and communicating trends, respiratory monitoring system 100 can notify user 102 of triggers.

In some examples, trend analysis module 258 uses a trend to determine one or more potential occurrences of breathing events, potential triggers, or the like. As one example, trend analysis module 258 may determine that a particular location is associated with an occurrence of a breathing event, and computing device 206 may generate a notification to user 102 when user 102 is near, approaching, or in the particular location. As another example, trend analysis module 258 may determine that one or more environmental parameters are associated with an occurrence of a breathing event, and computing device 206 may generate a notification to user 102 when the one or more environmental parameters are present at the location of user 102, when environmental information shows a trending toward the one or more environmental parameters, or the like. As another example, trend analysis module 258 may determine that past user input is associated with an occurrence of a breathing event, and computing device 206 may generate a notification to user 102 when user 102 provides the same or similar current user input to computing device 206. By determining a trend, respiratory monitoring system 100 may notify user 102 of a potential occurrence of a breathing event before a breathing event occurs. In this way, trend analysis module 258 may determine a notification that indicates a potential occurrence of a breathing event.

Computing device 206 may also generate a notification to user 102 identifying occurrences of breathing events over a period of time, ranking triggers (e.g., locations, one or more environmental parameters, user input, or the like), indicate a probability of a future occurrence of a breathing event, or the like. By basing notification on one or more trends, respiratory monitoring system 100 may more accurately determine at least one of an occurrence of a breathing event, a trigger associated with at least one occurrence of a breathing event, advice associate with at least one occurrence of a breathing event, at least one trigger, at least one trend, or the like.

In some examples, advice generation module 260 uses at least one of an occurrence of a breath event (e.g., obtained from respiratory parameter module 250), a location of user 102 (e.g., obtained from location information module 256), one or more environmental parameters (e.g., obtained from environmental information module 252), one or more triggers (e.g., obtained from trigger module 254), and one or more trends (e.g., obtained from trend analysis module 258) to determine advice. Advice may include any suitable information to assist user 102 in improving the respiratory function of user 102. For example, advice generation module 260 may determine a notification that indicates one or more locations, one or more environmental parameters, user input, one or more triggers, or the like are associated with occurrences of one or more breathing event. Advice generation module 250 may communicate advice to at least one of storage components 248 (e.g., at least one of respiratory parameter module 250, environmental information module 252, trigger module 254, location information module 256, and trend analysis module 258). By determining and communicating advice, respiratory monitoring system 100 can notify user 102 of advice.

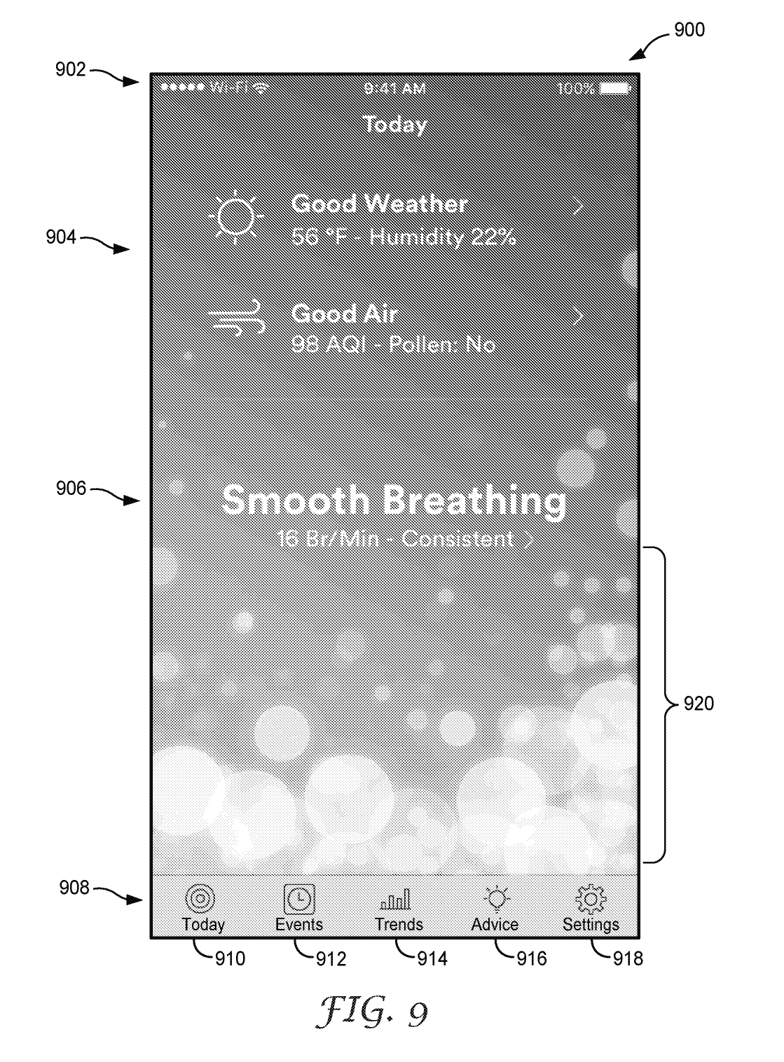

In some examples, computing device 206 may adjust (e.g., modify or update) detection of breathing events, trends and other indicators of respiratory function based on input from user 102. FIG. 7 is a flow diagram of an example technique for adjusting a detection algorithm based on input from a user 102. Although the technique of FIG. 7 will be described with respect to system 100 of FIG. 1 and computing device 206 of FIG. 2, in other examples, the technique of FIG. 7 may be performed using a different system, a different computing device, or both. Additionally, system 100 and computing device 206 may perform other techniques for receiving respiratory related parameters.

The technique illustrated in FIG. 7 includes, determining, by computing device 206, for example, one or more of respiratory parameter module 250, environmental information module 252, trigger module 254, location information module 256, trend analysis module 258, and advice module 258, at least one a trigger, a trend, and advice based on at least one of the occurrence of a breathing event, environmental information, location information, and a user input (702). For example, computing device 206 may apply a method to determine (e.g., a first algorithm) at least one a trigger, a trend, and advice based on at least one of the occurrence of a breathing event, environmental information, location information, and a user input.

After determining at least one a trigger, a trend, and advice (702), the technique of FIG. 7 includes receiving an input from user 102 (704). The user input is associated with at least one of the determined trigger, determined trend, and determined advice.

After receiving the second user input (704), the technique of FIG. 7 includes determining whether the determined trigger, determined trend, and determined advice is accurate based on the second user input (706). For example, the second user input may include an indication that at least one of a location and one or more environmental parameters was associated with an occurrence of a breathing event. In some examples, the second user input may indicate that the determined trigger, determined trend, and determined advice is accurate (YES branch). In other examples, the second user input may indicate that the determined trigger, determined trend, and determined advice is inaccurate (NO branch). In this way, the technique of FIG. 7 includes determining whether or not the first algorithm is accurate.

In examples in which the algorithm is accurate (YES branch), the algorithm may not be adjusted, and the technique may end (710).

In examples in which the algorithm is inaccurate (NO branch), computing device 206 may adjust the first algorithm based on the second user input to generate a second algorithm (708). After generating the second algorithm (708), the technique illustrated in FIG. 7 includes, again determining, by computing device 206, for example, one or more of respiratory parameter module 250, environmental information module 252, trigger module 254, location information module 256, trend analysis module 258, and advice module 258, at least one a trigger, a trend, and advice based on at least one of the occurrence of a breathing event, environmental information, location information, and a user input (repeating step 702). Computing device 206 may repeat steps 702, 706, and 708 n-times until the nth algorithm is accurate. By adjusting the algorithm based on second user input, respiratory monitoring system 100 may more accurately determine at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

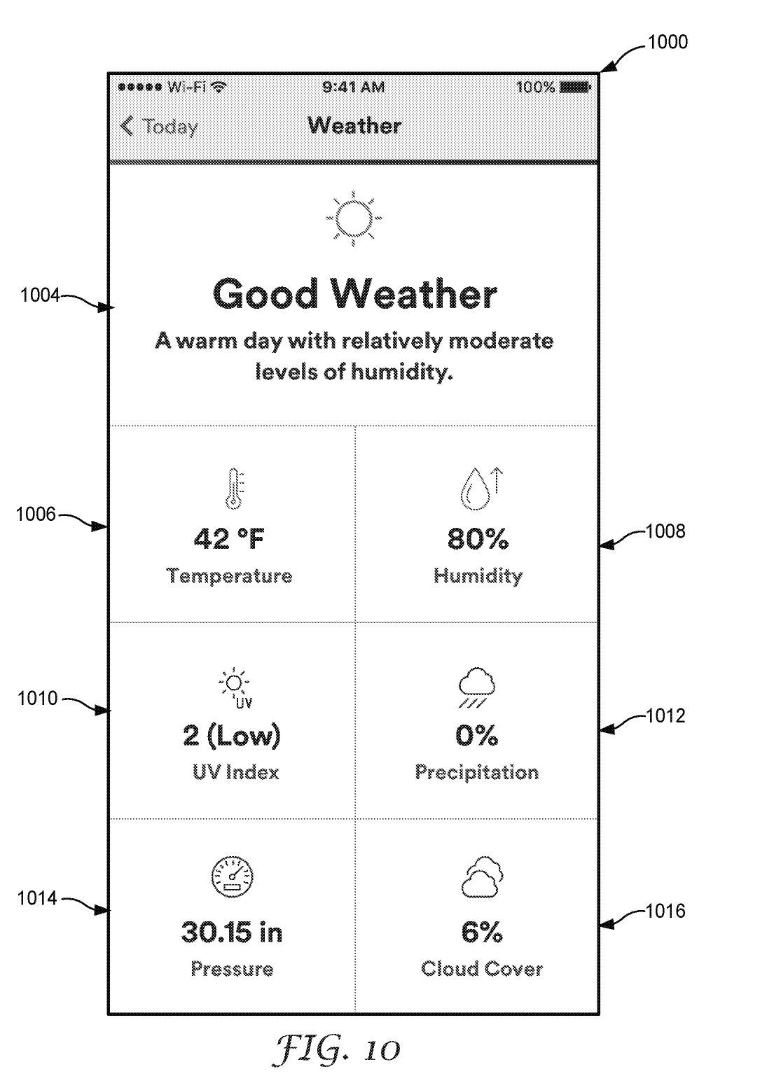

In some examples, the second user input may be received by computing device 206 in response to a notification provided to user 102. For example, FIG. 8 is a flow diagram of an example technique for adjusting the algorithm based on user input in response to providing user 102 a notification. Although the technique of FIG. 8 will be described with respect to system 100 of FIG. 1 and computing device 206 of FIG. 2, in other examples, the technique of FIG. 8 may be performed using a different system, a different computing device, or both. Additionally, system 100 and computing device 206 may perform other techniques for receiving respiratory related parameters.

The technique illustrated in FIG. 8 includes, determining, by computing device 206, for example, one or more of respiratory parameter module 250, environmental information module 252, trigger module 254, location information module 256, trend analysis module 258, and advice module 258, at least one a trigger, a trend, and advice based on at least one of the occurrence of a breathing event, environmental information, location information, and a user input (802). For example, as discussed above, computing device 206 may determine at least one of a trigger, a trend, and advice based on at least one of the occurrence of a breathing event, environmental information, location information, and a user input.

After determining at least one a trigger, a trend, and advice (802), the technique of FIG. 8 includes receiving, by user interface 208, a notification (804) (e.g., as discussed above with respect to step 602 of FIG. 6). After receiving the notification (802), the technique of FIG. 8 includes displaying, by display 201, the notification (806) (e.g., as discussed above with respect to step 604 of FIG. 6).

After displaying the notification (806), the technique of FIG. 8 includes receiving a second user input (808). The second user input may be the same or substantially similar to second user input discussed above with respect to step 704 of FIG. 7, except that here the second user input is regarding the accuracy of the notification. For example, the notification may prompt user 102 to indicate whether or not a trigger, a trend, or advice is accurate. In this way, the notification may include a survey for user 102 to provide second user input.

In response to receiving second user input, the technique of FIG. 8 includes determining whether the determined trigger, determined trend, and determined advice is accurate based on the second user input (810) (e.g., as discussed above with respect to step 706 of FIG. 7). In examples in which the algorithm is accurate (YES branch), the algorithm may not be adjusted, and the technique may end (814) (e.g., as discussed above with respect to step 710 of FIG. 7). In examples in which the algorithm is inaccurate (NO branch), computing device 206 may adjust the first algorithm based on the second user input to generate a second algorithm (812) (e.g., as discussed above with respect to step 708 of FIG. 7). By adjusting the algorithm based on second user input in response to a notification (e.g. a survey), respiratory monitoring system 100 may more accurately determine at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

### Examples

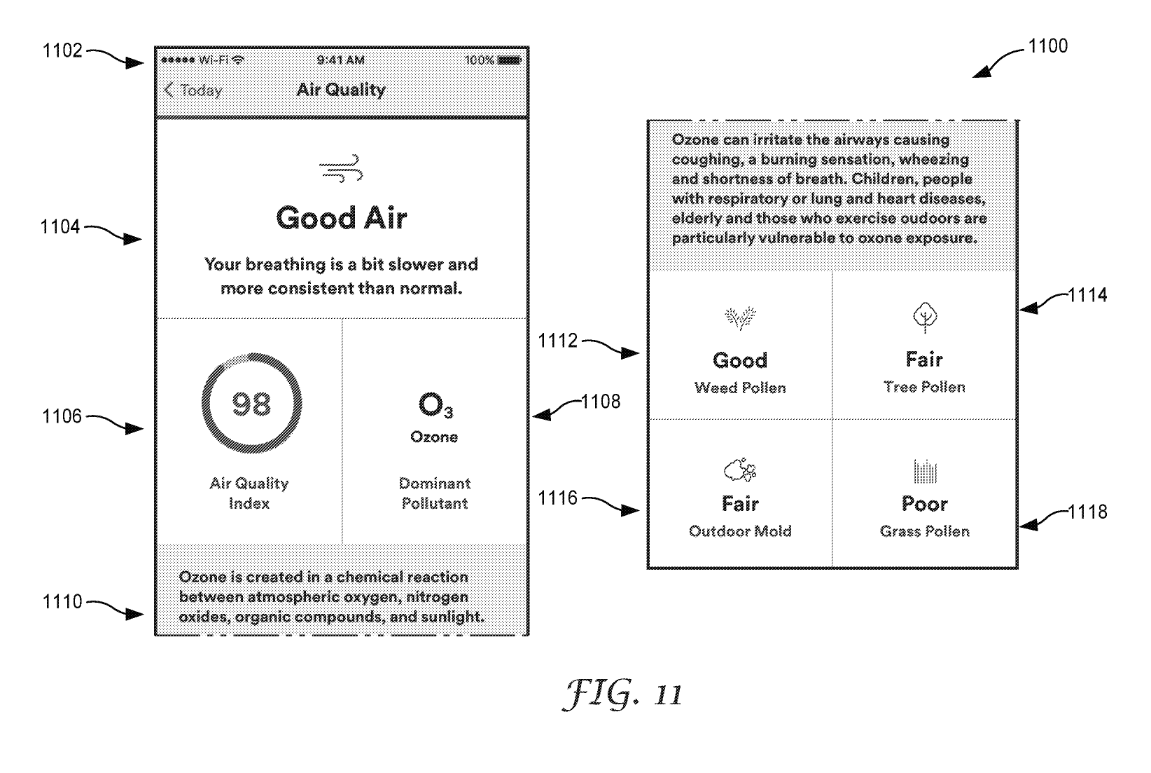

FIGS. 9-26 are schematic and conceptual diagrams illustrating various example displays of a user interface of a respiration monitoring system, in accordance with one or more aspects of the present disclosure. Although the example displays of FIGS. 9-26 will be described with respect to system 100 of FIG. 1 and computing device 206 of FIG. 2, in other examples, the technique of FIG. 8 may be performed using a different system, a different computing device, or both. Additionally, system 100 and computing device 206 may perform other techniques for receiving respiratory related parameters.

FIG. 9 is a schematic and conceptual diagram illustrating an example display of a today view 900 of a display 210 of respiration monitoring system 100. Today view 900 is titled “Today,” and includes a status bar 902, current environmental information 904, current breathing status 906, and navigation bar 908. Today view 900 may be displayed to user 102 when user 102 opens the respiratory monitoring application. Status bar 902 (e.g., a notification bar, a menu bar, or the like) includes network connectivity information, current time, and battery information. In some examples, status bar 902 includes location information, e.g., GPS connectivity or the like.

Current environmental information 904 includes current weather (e.g., a weather indicator, a current temperature, and a current humidity) and current air quality. In other examples, current environmental information 904 may include other environmental parameters, e.g., obtained from environmental information platform 126, as discussed above. In some examples, current environmental information 904 may be interactive. For example, computing device 206 may display detailed environmental information in response to selection by the user 102 (e.g., mouse click, finger tap, voice command, or the like) of the displayed current temperature, current humidity, or current air quality. In this way, current environmental information 940 may provide a notification to user 102 of environmental parameters associated with an occurrence of a breathing event.

Current breathing status 906 includes qualitative information (e.g., “smooth breathing”) and quantitative information (e.g., 16 breaths per minute (Br/Min) and consistent). In other examples, current breathing status 906 may include other respiratory data. In some examples, current breathing status 906 may be interactive. For example, computing device 206 may display detailed respiratory data in response to selection by the user 102 of the displayed qualitative information or quantitative information. In the example of FIG. 9, today view 900 includes bubbles 920. Bubbles 920 may include circles, cloud-like shapes, waves, or any other suitable geometry feature. Bubbles 920 rise and fall with the respiration of user 102. For example, computing device 206 may cause bubbles 920 to rise and fall relative to navigation bar 908 in response to an inhalation and exhalation, respectively, of user 102 sensed by sensor 104. In other examples, bubbles 920 may expand and contract, or otherwise change position or shape to indicate ventilation by user 102. In this way, current breathing status 906 may provide a notification to user 102 of the current respiratory parameters of user 102, an occurrence of a breathing event, or both.

Navigation bar 908 includes five navigation tiles: Today 910, Events 912, Trends 914, Advice 916, and Settings 918. The navigation tiles include text and a graphic. In other examples, the navigation tiles may include only text, only a graphic, or other features to indicate the relative location of the navigation tile. Navigation bar 908 enables user 102 to navigate to different views by selecting the navigation tiles. For example, computing device 206 may display a view indicated by a navigation tile in response to selection by the user 102 of the respective displayed navigation tile. In this way, user 102 may navigate the respiratory monitoring application.

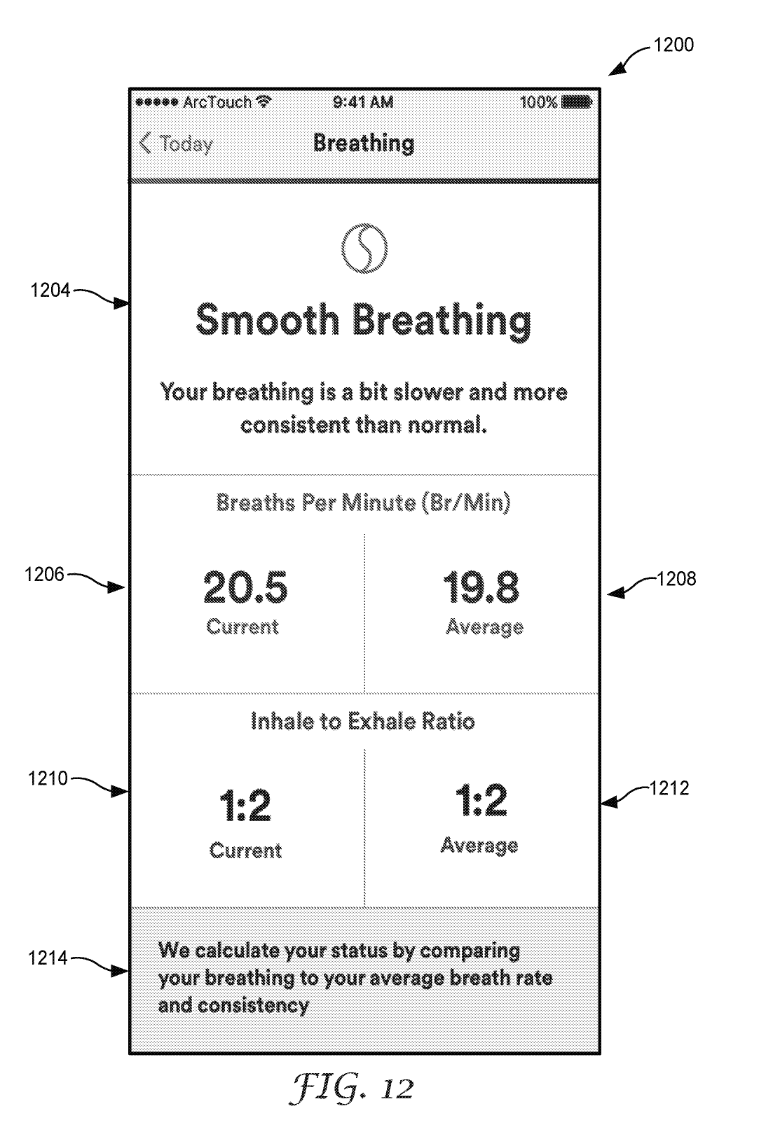

In response to selection by the user 102 of the displayed current weather (current environmental information 904 of FIG. 9), computing device 206 may display detailed environmental information including detailed current weather information. For example, FIG. 10 is a schematic and conceptual diagram illustrating an example display of a weather view 1000 of a display 210 of respiration monitoring system 100. Weather view 1000 is titled “Weather,” and includes a status bar 1002, weather summary 1004, and weather details 1006, 1008, 1010, 1012, and 1014. Weather view 1000 may be displayed to user 102 in response to user 102 selection of at least a portion of the current environmental information 904 of the today view 900 of FIG. 9.

Status bar 1002 may be the same or substantially similar to status bar 902 of FIG. 9. Weather summary 1004 includes a general indication of current weather conditions (e.g., “Good Weather”) and an indication of expected conditions (e.g., “A warm day with relatively moderate levels of humidity”). Weather details 1006, 1008, 1010, 1012, and 1014 include environmental parameters. The environmental parameters may include a graphic, a parameter value, and text (e.g., descriptors). For example, temperature detail 1006 includes a thermometer graphic, a temperature value of 42 degrees Fahrenheit, and the text “Temperature.” Humidity detail 1008 includes a droplet graphic with a trend increasing indicator arrow (i.e., humidity is rising), a humidity value of 80%, and the text “Humidity.” UV index detail 1010 includes a sun graphic, a UV index value of 2, and the text “UV Index.” Precipitation detail 1012 includes a rain cloud graphic, a precipitation value of 0%, and the text “Precipitation.” Barometric pressure detail 1014 includes a gauge graphic, a barometric pressure value of 30.15 in (e.g., inches of mercury, inHG, or “Hg), and the text “Pressure.” Cloud cover detail 1016 includes a cloud graphic, a cloud cover value of 6%, and the text “Cloud cover.” In other example, weather details may include different environmental parameters, fewer environmental parameters or additional environmental parameters, no graphics or different graphics, no parameter values or parameter values in any suitable units, or no text or additional text. By displaying weather details, weather view 1000 may provide to user 102 a notification of environmental information related to respiration of user 102 (e.g., environmental parameters associated with an occurrence of a breathing event, a trigger, or the like).

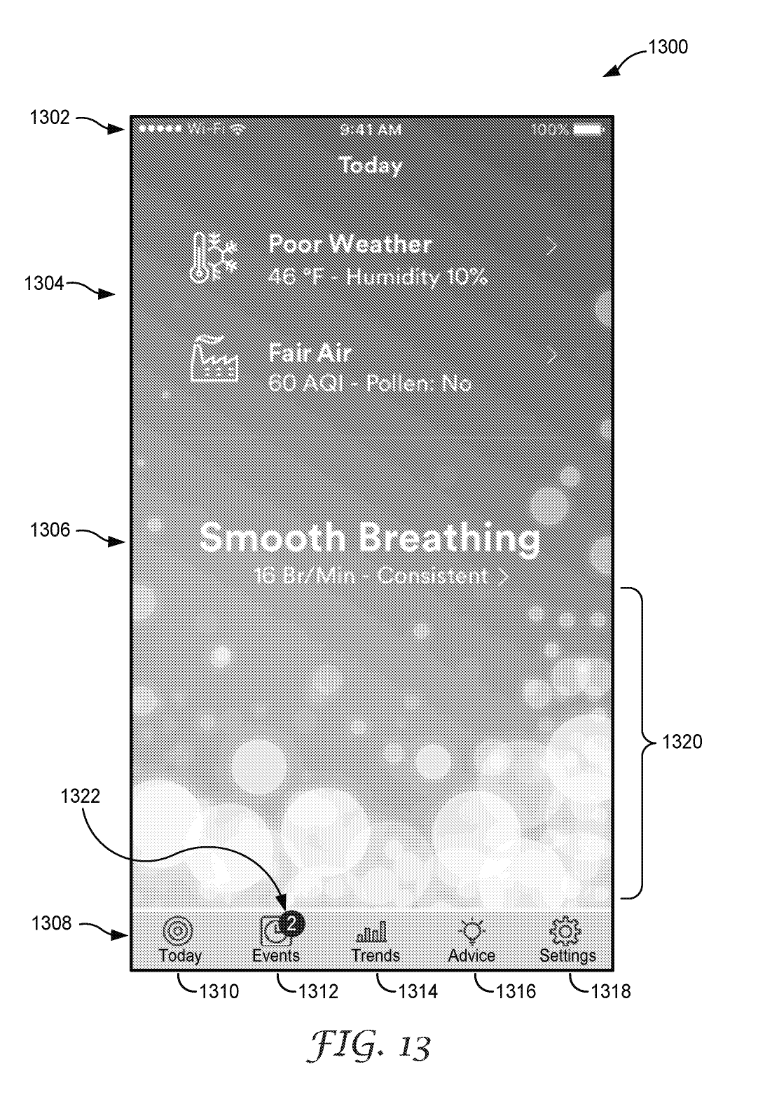

In response to selection by the user 102 of the displayed current air quality (current environmental information 904 of FIG. 9), computing device 206 may display detailed environmental information including detailed current air quality information. For example, FIG. 11 is a schematic and conceptual diagram illustrating an example display of an air quality view 1100 of a display 210 of respiration monitoring system 100. Air quality view 1100 is titled “Air Quality,” and includes a status bar 1102, air quality summary 1104, and air quality details 1106, 1108, 1110, 1112, 1114, 1116, and 1118. Air quality view 1100 may be displayed to user 102 in response to user 102 selection of at least a portion of the current environmental information 904 (e.g., current air quality) of the today view 900 of FIG. 9.

Status bar 1102 may be the same or substantially similar to status bar 902 of FIG. 9. Air quality summary 1104 includes a general indication of current air quality (e.g., “Good Air”) and an indication of expected air quality conditions or a respiration of user 102 (e.g., “Your breathing is a bit slower and more consistent than normal”). Air quality details 1106, 1108, 1110, 1112, 1114, 1116, and 1118 include environmental parameters related to air quality. The environmental parameters may include a graphic, a parameter value, and text (e.g., descriptors). For example, air quality index detail 1106 includes a gauge graphic, an air quality index of 89, and the text “Air Quality Index.” Dominant pollutant detail 1108 includes a molecular term symbol graphic (e.g., “O₃” for ozone), and the text “Ozone” and “Dominant Pollutant.” In some examples, air quality view 1100 includes definitions or advice based on one or more air quality details. For example, dominant pollutant definitions and advice 1110 may include “Ozone is created in a chemical reaction between atmospheric oxygen, nitrogen, organic compounds, and sunlight. Ozone can irritate the airways causing coughing, a burning sensation, wheezing and shortness of breath. Children, people with respiratory or lung and heart diseases, elderly and those who exercise outdoors are particularly vulnerable to ozone exposure.” Weed pollen detail 1112 includes a weed graphic, a qualitative weed pollen indicator “Good,” and the text “Weed Pollen.” Tree pollen detail 1114 includes a tree graphic, a qualitative tree pollen indicator “Fair”, and the text “Tree Pollen.” Outdoor mold detail 1116 includes a mold spore graphic, a qualitative outdoor mold pollen indicator “Fair”, and the text “Outdoor Mold.” Grass pollen detail 1118 includes a grass graphic, a qualitative grass pollen indicator “Poor”, and the text “Grass Pollen.” By displaying air quality details, air quality view 1100 may provide to user 102 a notification of environmental information related to air quality, potential triggers related to air quality, or both that affect the respiration of user 102 (e.g., environmental parameters associated with an occurrence of a breathing event, a trigger, or the like).

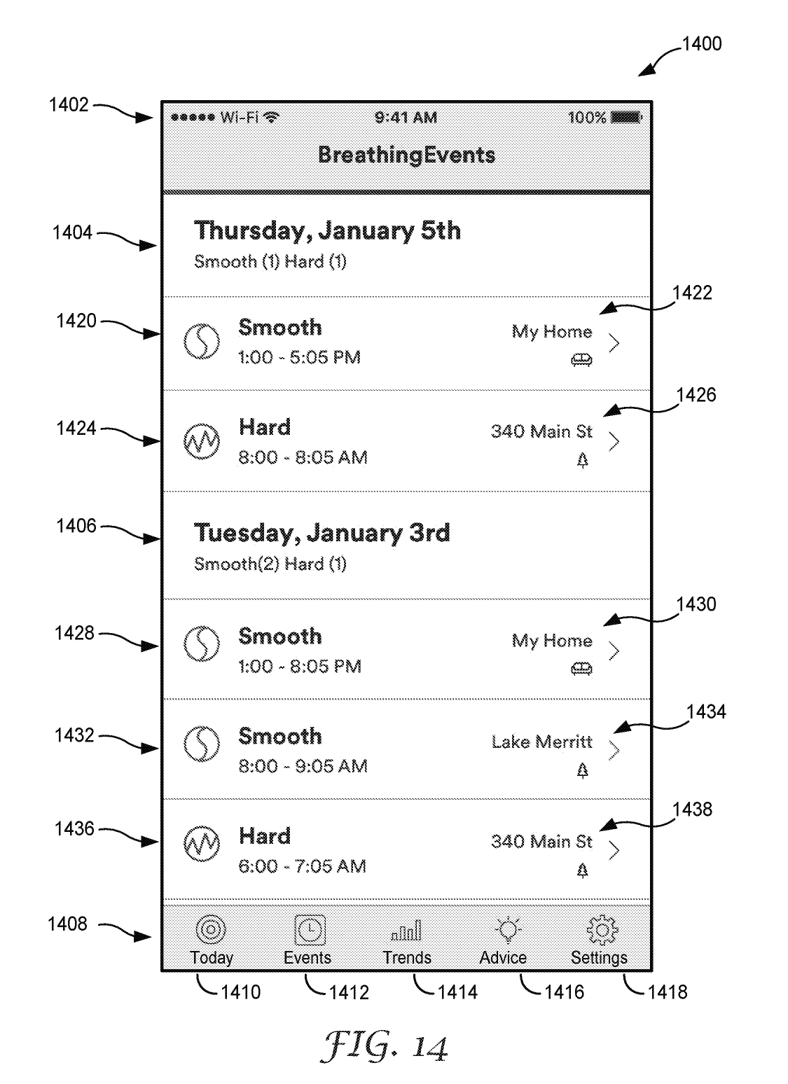

In response to selection by the user 102 of the displayed current breathing status 906 of FIG. 9, computing device 206 may display detailed respirator related parameters including detailed current and average respiratory parameters. For example, FIG. 12 is a schematic and conceptual diagram illustrating an example display of a breathing view 1200 of a display 210 of respiration monitoring system 100. Breathing view 1200 is titled “Breathing,” and includes a status bar 1202, breathing summary 1204, and respiratory related parameter details 1206, 1208, 1210, 1212, and 1214. Breathing view 1200 may be displayed to user 102 in response to user 102 selection of at least a portion of the current breathing status 906 of the today view 900 of FIG. 9.

Status bar 1202 may be the same or substantially similar to status bar 902 of FIG. 9. Breathing summary 1204 includes a general indication of current breathing status of user 102 (e.g., “Smooth Breathing” and “Your breathing is a bit slower and more consistent than normal”). In this way, breathing summary 1204 may indicate an occurrence of a breathing event. Respiratory related parameter details 1206, 1208, 1210, 1212, and 1214 include respiratory parameters of user 102. The respiratory parameters may include a parameter value and text (e.g., descriptors). For example, breaths per minute details 1206 and 1208 include, respectively, current breaths per minute (Br/Min) of 20.5 and an average Br/Min of 19.8. The inhale to exhale ratio details 1210 and 1212 include, respectively, a current inhale to exhale ratio of 1:2 and an average inhale to exhale ratio of 1:2. Breathing status calculation details 1214 indicate how the breathing status is determined, e.g., “We calculate your status by comparing your breathing to your average breath rate and consistency.” Additionally, or alternatively, breathing view 1200 may indicate a duration of an occurrence of a breathing event, such as, for example, “event duration 2-minute streak detected.” In other example, breathing status details may include different respiratory related parameters, fewer respiratory related parameters or additional respiratory related parameters, graphics, no parameter values or parameter values in any suitable units, or no text or additional text. By displaying respiratory related parameter details, breathing view 1200 may provide to user 102 a notification of respiratory related parameters to user 102 (e.g., respiratory related parameters associated with an occurrence of a breathing event, a trigger, or the like).

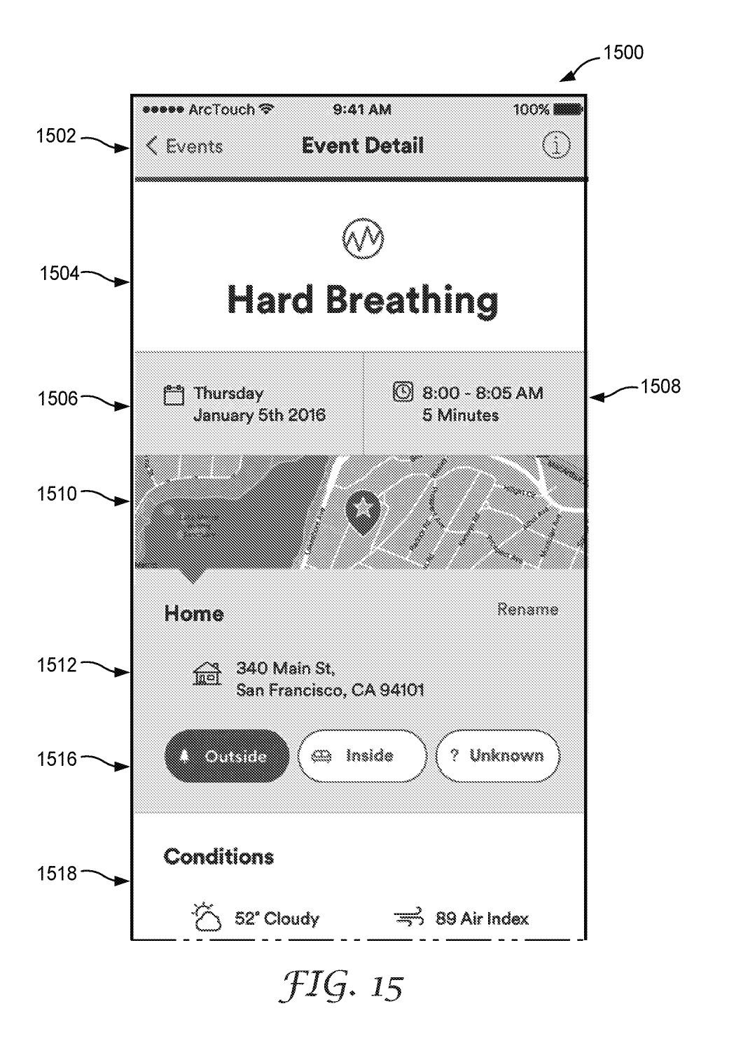

In response to determining a notification, computing device 206 may cause display 210 to display a notification to user 102. For example, FIG. 13 is a schematic and conceptual diagram illustrating an example display of a notification on a today view 1300 of display 210 of respiration monitoring system 100. Today view 1300 may be the same or substantially similar to today view 900 of FIG. 9, except for the differences describe herein. For example, similar to today view 900 of FIG. 9, today view 1300 includes a status bar 1302, current environmental information 1304, current breathing status 1306, navigation bar 1308 that includes five navigation tiles (e.g., Today 1310, Events 1312, Trends 1314, Advice 1316, and Settings 1318), and bubbles 1320.

As shown in FIG. 13, in response to determination of an occurrence of a breathing event, computing device 206 causes display 210 to display a notification to user 102 as a number badge 1322 over Events 1312 navigation tile. Number badge 1322 includes a red circle with the numeral “2” displayed over a portion of Events 1312. The numeral “2” indicates that computing device 206 recorded two occurrences of breathing events, e.g., since user 102 has viewed the events page. In other examples, number badge 1322 may be displayed over other navigation tiles or other portions of today view 1300, such as, for example, over Trends 1314, Advice 1316, or the like. Additionally, number badge 1322 may include other numerals to indicate one or more notifications to user 102. By displaying number badge 1322, respiratory monitoring system 100 provides to user 102 a notification of an occurrence of a breathing event, a trigger, a trend, advice, or the like.

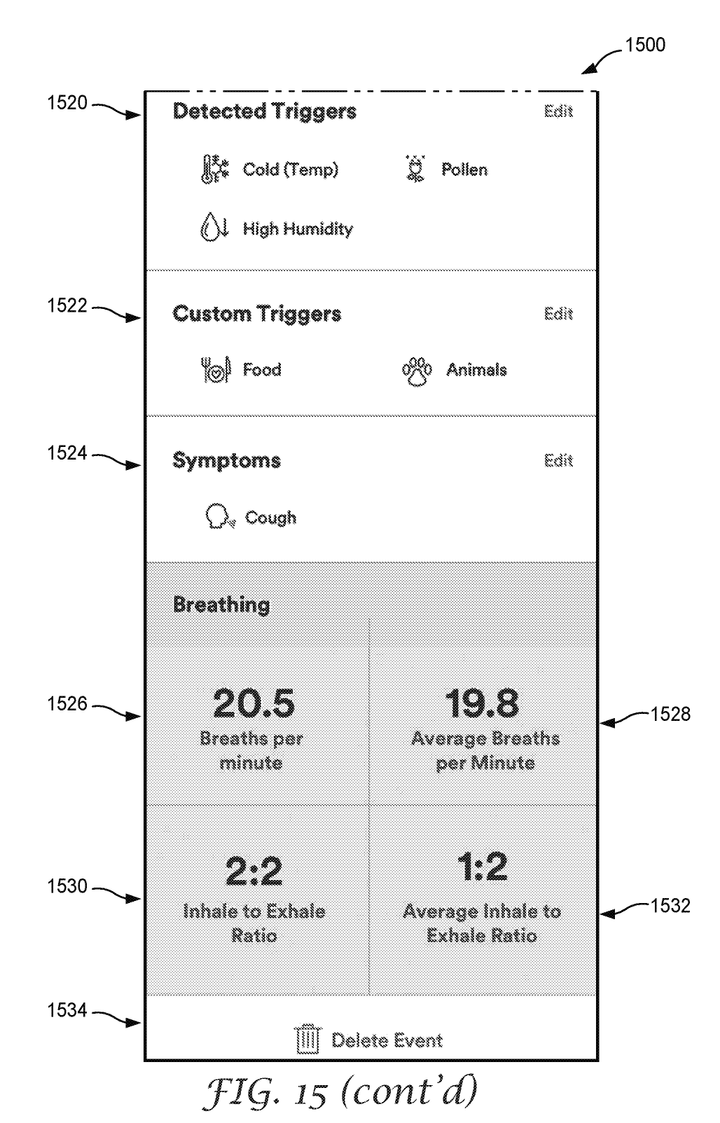

In response to selection by the user 102 of Events 912 navigation tile of FIG. 9, computing device 206 may display a breathing events view. For example, FIG. 14 is a schematic and conceptual diagram illustrating an example display of breathing events view 1400 on display 210 of respiration monitoring system 100. Breathing events view 1400 is titled “Breathing Events,” and includes a status bar 1402, a first list of breathing events 1404, a second list of breathing events 1406, and navigation bar 1408 that includes five navigation tiles (e.g., Today 1410, Events 1412, Trends 1414, Advice 1416, and Settings 1418).

Status bar 1402 and navigation base 1408 may be the same or substantially similar to status bar 902 and navigation bar 908, respectively, of FIG. 9. As shown in FIG. 14, first list of breathing events 1404 includes a date (e.g., Thursday, January 5th) and an indication of the number of smooth breathing events and hard breathing event that occurred on that date (e.g., Smooth (1), Hard (1)). (The numerals with respect to the list of breathing events are referential, not ordinal.) Similarly, second list of breathing events 1406 includes a date (e.g., Tuesday, January 3rd) and an indication of the number of smooth breathing events and hard breathing event that occurred on that date (e.g., Smooth (2), Hard (1)). Although the date in first and second lists of breathing events 1404 and 1406 include one day, in other example, a respective list of breathing events may include a week, a month, a year, or any suitable duration. In some examples, fewer or more than two lists of breathing events may be displayed.

Each occurrence of a breathing event is displayed in chronological order within the respective first and second lists of breathing events. Each occurrence of a breathing event includes a graphic and indicator of the type of breathing event, a duration or timeframe of the occurrence of the breathing event, and a location of the breathing event. Generally, any suitable text or graphics may be used to provide a notification to user 102 of the occurrences of breathing events. For example, first list of breathing events 1404 includes first smooth breathing event 1420 and first hard breathing event 1424. First smooth breathing event 1420 includes a text indication of a smooth breathing event and a smooth breathing graphic, a timeframe of the event (e.g., 1:00-5:05 PM), and a location 1422 of the breathing event (e.g., My Home) with a graphic of a couch. First hard breathing event 1424 includes a text indication of a hard breathing event and a hard breathing graphic, a timeframe of the event (e.g., 8:00-8:05 AM), and a location 1426 of the breathing event (e.g., 340 Main St.) with a graphic of a tree. Second list of breathing events 1406 includes second smooth breathing event 1428, third smooth breathing event 1432, and second hard breathing event 1436. Second smooth breathing event 1428 includes a text indication of a smooth breathing event and a smooth breathing graphic, a timeframe of the event (e.g., 1:00-8:05 PM), and a location 1430 of the breathing event (e.g., My Home) with a graphic of a couch. Third smooth breathing event 1432 includes a text indication of a smooth breathing event and a smooth breathing graphic, a timeframe of the event (e.g., 8:00-9:05 AM), and a location 1434 of the breathing event (e.g., Lake Merritt) with a graphic of a tree. Second hard breathing event 1424 includes a text indication of a hard breathing event and a hard breathing graphic, a timeframe of the event (e.g., 6:00-7:05 AM), and a location 1438 of the breathing event (e.g., 340 Main St.) with a graphic of a tree. By displaying lists of breathing events, respiratory monitoring system 100 provides user 102 a notification of an occurrence of a breathing event. Providing user 102 a notification of the occurrence of a breathing event enables user 102 to track occurrences of breathing events to identify triggers, trends, or the like.

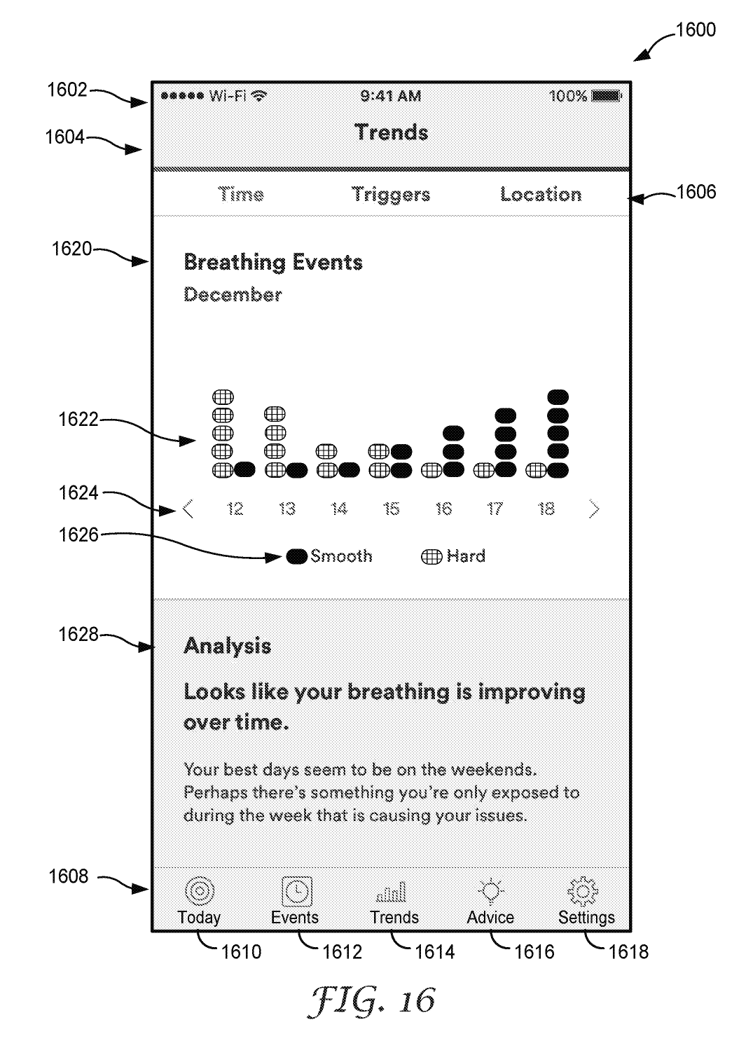

In response to selection by the user 102 of a breathing event from a list of occurrences of breathing events, computing device 206 may display breathing events details. For example, FIG. 15 is a schematic and conceptual diagram illustrating an example display of event details view 1500 on display 210 of respiration monitoring system 100. Event details view 1500 is titled “Event Details,” and includes a status bar 1502, an event type 1404, an event date 1506, an event duration 1508, event location data including a map 1510 and location details 1512, a location survey 1516, environmental parameters 1518, detected triggers 1520, custom triggers 1522, event symptoms 1524, event breathing status 1526, 1528, 1530, and 1532, and a delete option 1534.

Status bar 1502 may be the same or substantially similar to status bar 902 of FIG. 9. As shown in FIG. 15, event type 1404 provides an indication of the type of breathing event (e.g., hard breathing or smooth breathing) for the selected occurrence of a breathing event. Event date 1506 provides an indication of a date of the occurrence of the breathing event (e.g., Thursday, Jan. 5, 2016). Event duration 1508 provides an indication of a timeframe and a duration of the occurrence of the breathing event (e.g., 8:00-8:05 AM, 5 Minutes).

Map 1510 provide an indication of the location of the occurrence of the breathing event on a map. Location details 1512 provide an indication of a named location (e.g., named by input from user 102) and address of the location (e.g., Home, 340 Main St., San Francisco, Calif. 94101). Location survey 1516 provides user 102 a survey (e.g., as discussed above with respect to FIG. 8) for user 102 to input additional location information. For example, location survey 1516 includes three options: Outside, Inside, and Unknown. User 102 may select one of the three options to provide to computing device 206 user input regarding the location information.

Environmental parameters 1518 provide an indication of the environmental parameters determined to be present during the occurrence of the breathing event. For example, environmental parameters 1518 include weather information (e.g., partly cloudy graphic, 52° F., and Cloudy) and air quality index information (e.g., 89 Air Index).

Detected triggers 1520 include triggers determined by computing device 206 to be associated with the occurrence of the breathing event. For example, detected triggers 1520 include Cold (Temp), Pollen, and High Humidity. Custom triggers 1522 include triggers determined by user 102 to be associated with the occurrence of the breathing event. Computing devise 206 by receive from user 102 user input (e.g., second user input as discussed in FIGS. 7 and 8) indicating custom triggers 1522. For example, custom triggers 1522 include Food and Animals. As shown in FIG. 15, each of respective detected triggers 1520 and custom triggers include a graphic. In some examples, detected triggers 1520 and custom triggers 1522 are editable by user input. For example, user 102 may select the “Edit” tile to add detected triggers or custom triggers, delete detected triggers or custom triggers, make notes with respect to detected triggers or custom triggers, or the like.

Event symptoms 1524 provide an indication of symptoms experienced user 102 that are associated with the occurrence of the breathing event. For example, event symptoms 1524 include cough. As shown in FIG. 15, a respective symptom 1524 includes a respective graphic.

Event breathing status 1526, 1528, 1530, and 1532, provide an indication of respiratory related parameters associated with the occurrence of the breathing event. For example, breaths per minute details 1526 and 1528 include, respectively, current breaths per minute (Br/Min) of 20.5 and an average Br/Min of 19.8. The inhale to exhale ratio details 1530 and 1532 include, respectively, a current inhale to exhale ratio of 2:2 (e.g., 1:1) and an average inhale to exhale ratio of 1:2.

Delete option 1534 provides user 102 an option to delete the occurrence of the breathing event.

By displaying event details, respiratory monitoring system 100 provides user 102 a notification of an occurrence of a breathing event and enables user 102 to provide user input, which computing device 206 may use to adjust one or more algorithms used to determine the occurrence of a breathing event. In this way, event detail view 1500 respiratory monitoring system 100 may more accurately determine at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

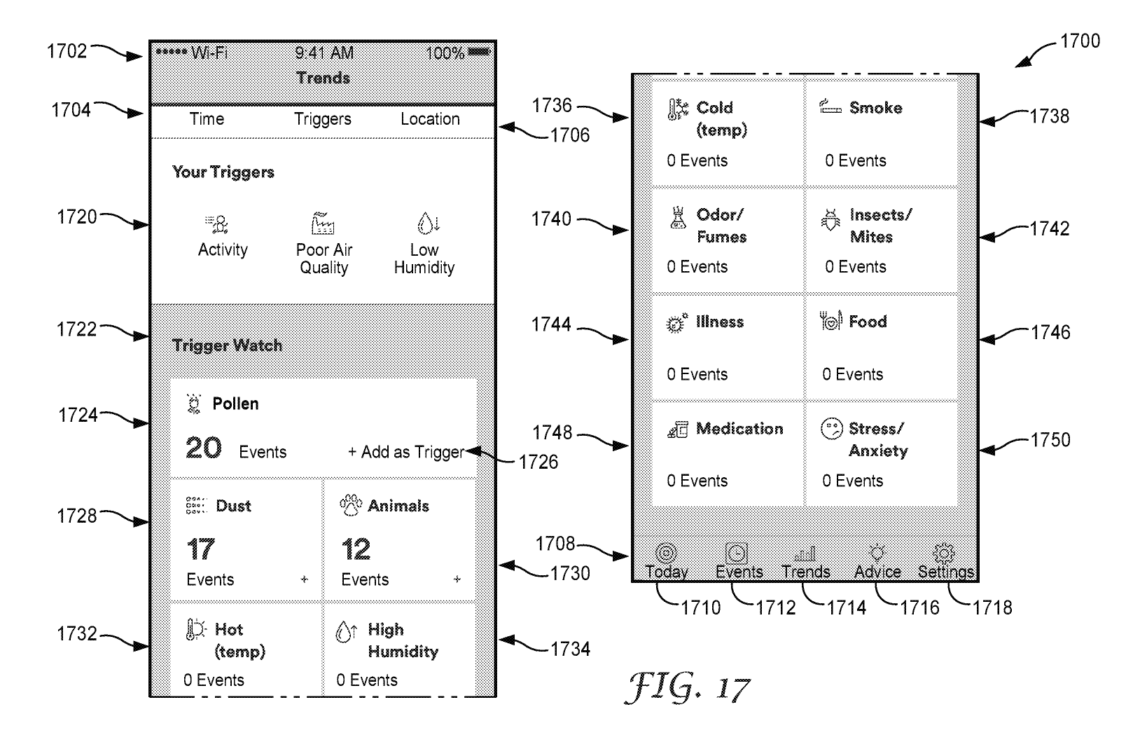

In response to selection by the user 102 of Trends 914 in navigation bar 908 of FIG. 9, computing device 206 may display a trends view. For example, FIG. 16 is a schematic and conceptual diagram illustrating an example display of trends view 1600 on display 210 of respiration monitoring system 100. Trends view 1600 is titled “Trends,” and includes a status bar 1602, a trend 1604, and navigation bar 1608 that includes five navigation tiles (e.g., Today 1610, Events 1612, Trends 1614, Advice 1616, and Settings 1618).

Status bar 1602 and navigation base 1608 may be the same or substantially similar to status bar 902 and navigation bar 908, respectively, of FIG. 9. As shown in FIG. 16, trend 1604 includes a trend category bar 1606. Trend categories include Time, Triggers, and Location. For example, as discussed above, computing device 206 may determine a trend based on one or more of a plurality of occurrences of breathing events (e.g., number of occurrences of breathing events), determined triggers, and location information. In other examples, trend categories may include fewer or additional categories. As shown in FIG. 16, trend 1604 displays occurrences of breathing events 1620 in the Time category for the month of August. Trend graphic 1622 displays as stacked blocks the number of occurrences of breathing events (e.g., smooth and hard) for each respective date of date bar 1624 (e.g., August 12th through 18th). For example, August 12th shows five hard breathing events and one smooth breathing event. Date bar 1624 includes selectable indicators to scrolls through the dates. Trend graphic 1622 includes legend 1626 to indicate the graphic used for each of hard breathing events (e.g., patterned blocks) and smooth breathing events (e.g., solid blocks). Any suitable color, pattern, or other distinguishing features may be sued to indicate different types of breathing events. In some examples, trend 1604 includes analysis 1628. Analysis 1628 includes a description of the trend displayed in trend 1604 and advice. For example, analysis 1628 describes “Looks like your breathing is improving over time. Your best days seems to be on the weekends. Perhaps there's something you're only exposed to during the week that is causing your issues.” In other examples, analysis 1628 may include different descriptions of trend 1604, different advice, or both. By displaying trends view 1600, respiratory monitoring system 100 provides user 102 a notification of at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

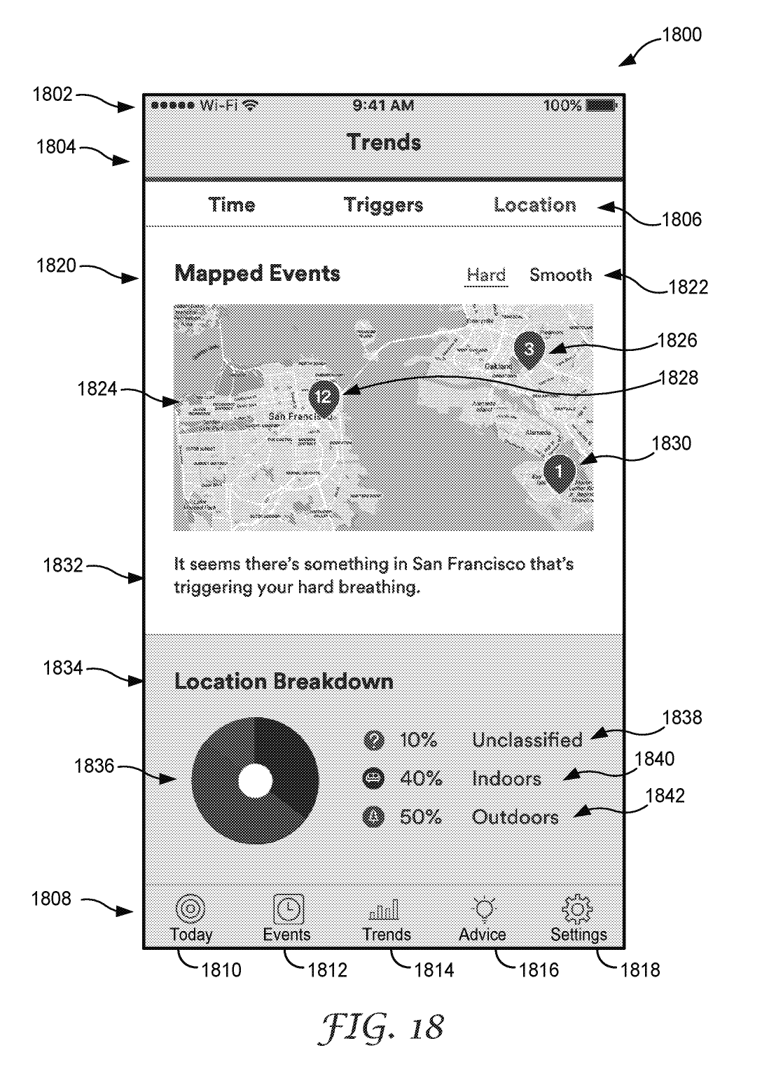

In response to selection by the user 102 of Triggers in trends bar 1606 of trends view 1600 of FIG. 16, computing device 206 may display a trigger trends view. For example, FIG. 17 is a schematic and conceptual diagram illustrating an example display of trigger trends view 1700 on display 210 of respiration monitoring system 100. Trigger trends view 1700 is titled “Trends,” and includes a status bar 1702, a trigger trend 1704, and navigation bar 1708 that includes five navigation tiles (e.g., Today 1710, Events 1712, Trends 1714, Advice 1716, and Settings 1718).

Status bar 1702 and navigation base 1708 may be the same or substantially similar to status bar 902 and navigation bar 908, respectively, of FIG. 9. As shown in FIG. 17, trigger trend 1604 includes a trend category bar 1706. Trend category bar 1706 may be the same or substantially similar to trend category base 1606 of FIG. 16. Trigger trend 1704 provides an indication of determined triggers 1720 for user 102 (e.g., Your Triggers) and determined potential triggers 1722 (e.g., Trigger Watch). Determined triggers 1720 includes triggers determined by computing device 206 to be associated with the occurrence of a plurality of breathing events. For example, as shown in FIG. 17, Your Triggers includes activity, poor air quality, and low humidity, and a respective graphic for each trigger. Determined potential triggers 1722 include triggers determined by computing device 206 to be potentially associated with the occurrence of a plurality of breathing events. Each potential trigger may include a number of occurrences of breathing events associated with the respective trigger, an option for user 102 to add a respective determined potential trigger 1722 to determined triggers 1720, and a respective graphic. For example, as shown in FIG. 17, Trigger Watch includes pollen trigger 1724. Pollen trigger 1724 includes a graphic of a flower (and the text “Pollen”), an indication that pollen was determined to be associated with 20 occurrences of breathing events, and a selectable “+ Add as Trigger” button. Dust trigger 1728 includes a graphic of dust (and the text “Dust”), an indication that dust was determined to be associated with 17 occurrences of breathing events, and a selectable “+” button. Animals trigger 1730 includes a graphic of a paw print (and the text “Animals”), an indication that dust was determined to be associated with 12 occurrences of breathing events, and a selectable “+” button. Trigger Watch includes other potential triggers not yet associated with an occurrence of a breathing event, including Hot (temp) 1732, High Humidity 1734, Cold (temp) 1736, Smoke 1738, Odor/Fumes 1740, Insects/Mites 1742, Illness 1744, Food 1746, Medication 1748, and Stress/Anxiety 1750. In other examples, different determined triggers, determined potential triggers, or both may be displayed in trigger trends view 1700. In other examples, trigger trends view 1700 may include different or additional graphics such as bar charts, pie charts, or the like. By displaying trigger trends view 1700, respiratory monitoring system 100 provides user 102 a notification of at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

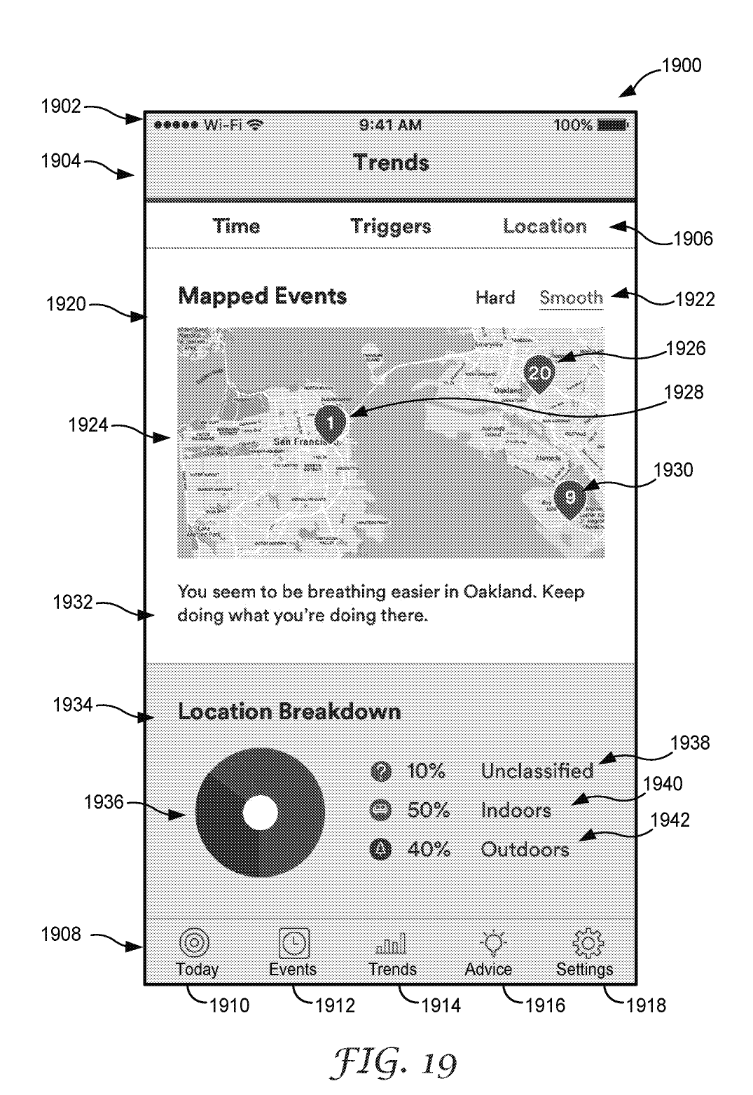

In response to selection by the user 102 of Location in trends bar 1606 of trends view 1600 of FIG. 16, computing device 206 may display a location trends view. For example, FIG. 18 is a schematic and conceptual diagram illustrating an example display of location trends view 1800 on display 210 of respiration monitoring system 100. Location trends view 1800 is titled “Trends,” and includes a status bar 1802, a location trend 1804, and navigation bar 1808 that includes five navigation tiles (e.g., Today 1810, Events 1812, Trends 1814, Advice 1816, and Settings 1818).

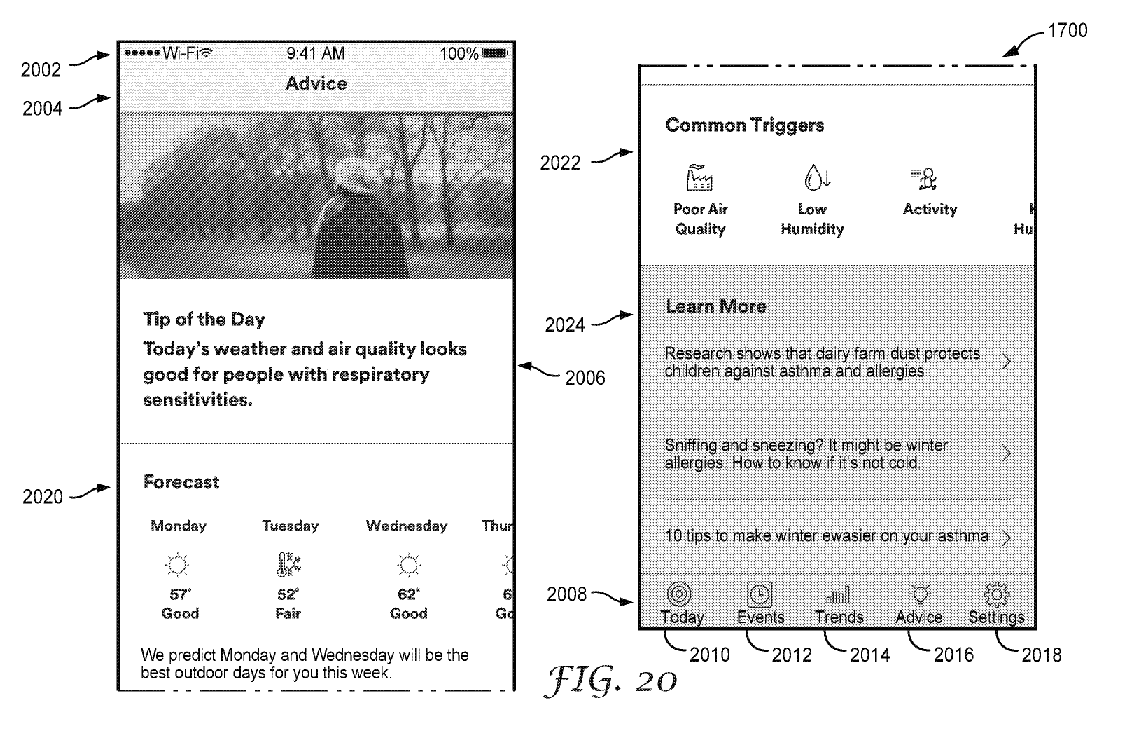

Status bar 1802 and navigation base 1808 may be the same or substantially similar to status bar 902 and navigation bar 908, respectively, of FIG. 9. As shown in FIG. 18, trigger trend 1804 includes a trend category bar 1806. Trend category bar 1806 may be the same or substantially similar to trend category base 1606 of FIG. 16. Location trend 1804 provides an indication of determined locations 1820 of user 102 during occurrences of hard breathing events (e.g., Mapped Events) and categories of determined locations 1834 (e.g., Location Breakdown). Determined locations 1820 includes locations determined by computing device 206 to be associated with an occurrence of a hard breathing event. Determined locations 1820 includes a selectable indicator 1822 to display hard breathing events on map 1824. In some examples, user 102 may select “Smooth” to display determined locations of smooth breathing events (see FIG. 19, discussed below). Map 1824 includes indicators of locations of occurrences of hard breathing events 1826, 1828, and 1830. Indicators of locations of occurrences of hard breathing events 1826, 1828, and 1830 include a numeral indicating the number of occurrences of breathing events associated with a respective location. Determined locations 1820 may include a description of the determined locations 1832. For example, description of the determined locations 1832 provides “It seems there's something in San Francisco that's triggering your hard breathing.” In other examples, description of the determined locations 1832 may include other descriptions of the determined locations 1820.

Categories of determined locations 1834 includes a graphic representing a proportion of occurrences of hard breathing events determined for each of a location category. For example, as shown in FIG. 18, pie chart 1836 is shown for each of three location categories. The location categories include Unclassified 1838, Indoors 1840, and Outdoors 1842. In other examples, additional location categories or different location categories may be used.

By displaying location trends view 1800, respiratory monitoring system 100 provides user 102 a notification of at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

As discussed above, in response to selection by the user 102 of the “Smooth” selectable indicator 1822 of FIG. 18, computing device 206 may display a location trends view for smooth breathing events. For example, FIG. 19 is a schematic and conceptual diagram illustrating an example display of location trends view 1900 on display 210 of respiration monitoring system 100. Location trends view 1900 is the same or substantially similar to location trends view 1800 discussed above with respect to FIG. 18, except for the differences describe herein.

For example, like location trends view 1800, location trends view 1900 is titled “Trends,” and includes a status bar 1902, a location trend 1904, a trend category bar 1906, and navigation bar 1908. Location trend 1904 provides an indication of determined locations 1920 of user 102 during occurrences of smooth breathing events (e.g., Mapped Events) and categories of determined locations 1934 (e.g., Location Breakdown). Determined locations 1920 includes a selectable indicator 1922. Map 1924 includes indicators of locations of occurrences of smooth breathing events 1926, 1928, and 1930. Indicators of locations of occurrences of smooth breathing events 1926, 1928, and 1930 include a numeral indicating the number of occurrences of breathing events associated with a respective location. Determined locations 1920 may include a description of the determined locations 1932. For example, description of the determined locations 1932 provides “You seem to be breathing easier in Oakland. Keep doing what you're doing there.” In other examples, description of the determined locations 1932 may include other descriptions of the determined locations 1920.

Categories of determined locations 1934 includes a graphic representing a proportion of occurrences of smooth breathing events determined for each of a location category. For example, as shown in FIG. 19, pie chart 1936 is shown for each of three location categories. The location categories include Unclassified 1938, Indoors 1940, and Outdoors 1942. In other examples, additional location categories or different location categories may be used.

By displaying location trends view 1900, respiratory monitoring system 100 provides user 102 a notification of at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

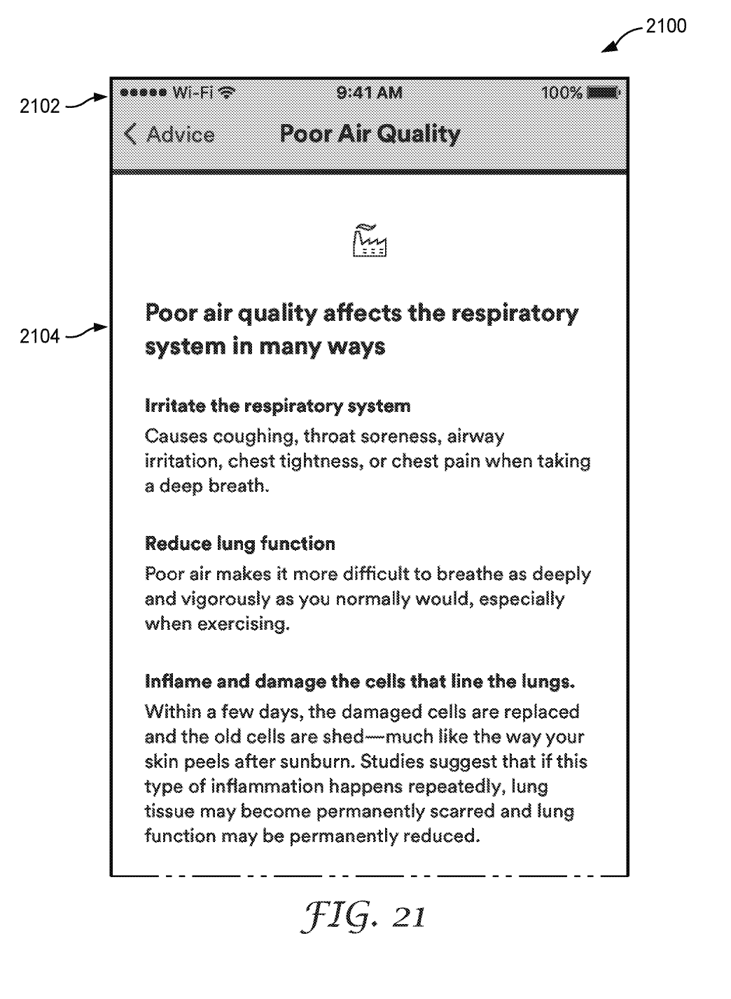

In response to selection by the user 102 of Advice 914 in navigation bar 908 of FIG. 9, computing device 206 may display an advice view. For example, FIG. 20 is a schematic and conceptual diagram illustrating an example display of advice view 2000 on display 210 of respiration monitoring system 100. Advice view 2000 is titled “Advice,” and includes a status bar 2002, advice details 2004, and navigation bar 2008 that includes five navigation tiles (e.g., Today 2010, Events 2012, Trends 2014, Advice 2016, and Settings 2018). Status bar 2002 and navigation base 2008 may be the same or substantially similar to status bar 902 and navigation bar 908, respectively, of FIG. 9. As shown in FIG. 16, advice details 1604 includes a photo, a tip of the day 2006, a forecast 2020, common triggers 2022, and learn more section 2024.

Tip of the day 2006 may include any suitable information, such as, for example, information relevant to respirator health. For example, tip of the day 2006 provides “Today's weather and air quality looks good for people with respiratory sensitivities.” In other examples, tip of the day 2006 may include other information, such as, for example, poor air quality warnings, weather advisories, potential triggers, trends, or the like.

Forecast 2020 includes environmental information for respective days of the week. For example, includes environmental information including temperature and air quality for Monday (e.g., 57° F., good air quality), Tuesday (e.g., 52° F., fair air quality), and Wednesday (e.g., 62° F., good air quality). In some examples, forecast 2020 includes a weather prediction, such as, for example, an upcoming day that has good outdoor air quality. For example, forecast 2020 provides “We predict Monday and Wednesday will be the best outdoor days for you this week.” In other examples, forecast 2020 may include other weather predictions related to respiratory health.

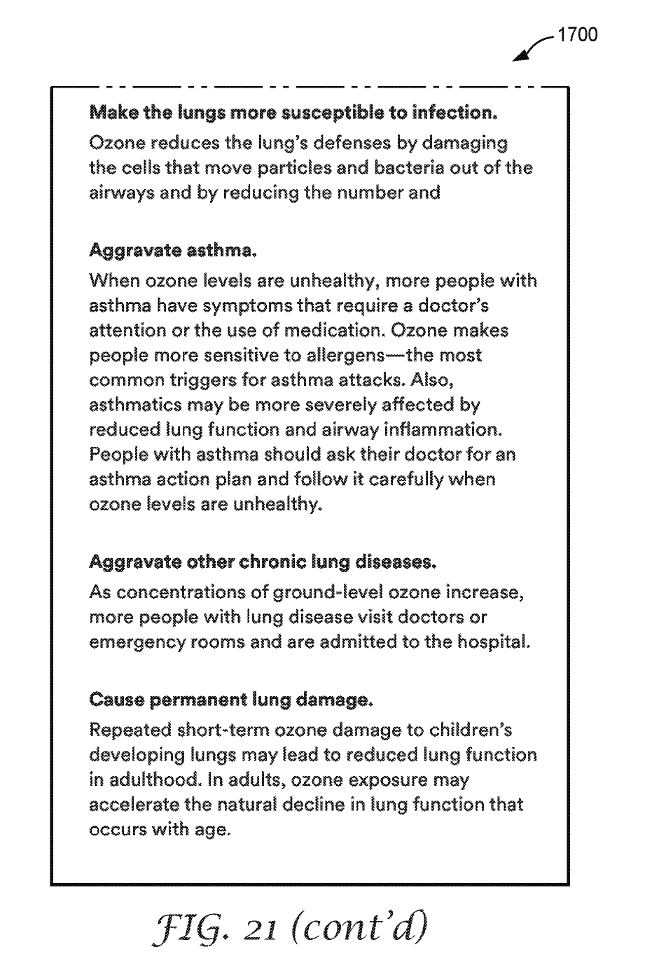

Common triggers 2022 includes a list of common triggers of occurrences of breathing events for user 102. For example, common triggers 2022 includes the three determined triggers 1720 of FIG. 17, including poor air quality, low humidity, and activity. In other examples, common triggers 2022 may include triggers of hard breathing events, triggers of smooth breathing events, or both. In some examples, each respective determined trigger may be selectable by user 102 to cause computing device 206 to display additional information related to the respective determined trigger (e.g., FIG. 21).

Learn more section 2024 includes any suitable additional advice related to respiratory health. For example, learn more section 2024 includes three sections 2026, 2028, and 2030, each respective section selectable by user 102 to cause computing device 206 to display additional information related to the respective section. First section 2026 provides “Research shows that dairy farm dust protects children against asthma and allergies.” Second section 2028 provides “Sniffing and sneezing? It might be winter allergies. How to know if it's not a cold.” Third section 2030 provides “10 Tips to make winter easier on your asthma.”

By displaying advice view 2000, respiratory monitoring system 100 provides user 102 a notification of advice associate with at least one occurrence of a breathing event, at least one trigger, at least one trend, or the like. The notification of advice may enable user 102 to improve control of occurrences of breathing events.

As discussed above, each respective determined trigger of common trigger 2022 may be selectable by user 102 to cause computing device 206 to display additional information related to the respective determined trigger. For example, FIG. 21 is a schematic and conceptual diagram illustrating an example display of determined trigger view 2100 on display 210 of respiration monitoring system 100. Determined trigger view 2100 is titled “Poor Air Quality.” In other examples, determined trigger view 2100 may include any suitable title representative of the type of determined trigger. Determined trigger view includes a status bar 2102, determined trigger details 2104. Status bar 2102 may be the same or substantially similar to status bar 902 of FIG. 9. In some examples, determined trigger view may include a navigation bar, such as, for example, the same or similar to navigation bar 908 of FIG. 9.

As shown in FIG. 21, determined trigger details 2104 includes trigger information related to the respective determined trigger. The trigger information may include any suitable information related to the respective determined trigger. For example, determined trigger details 2104 may provide how the determined trigger may affect respiration (e.g., “Poor air quality affect the respiratory system in many ways. Irritate the respiratory system: Causes coughing, throat soreness, airway irritation, chest tightness, or chest pain when taking a deep breath.”). Determine trigger details 2104 also may provide symptoms associated with the determined trigger (e.g., “Reduce lung function: Poor air make is more difficult to breath as deeply and vigorously as you normally would, especially when exercising.). Determine trigger details 2104 also may provide biophysical effects of exposure to a trigger (e.g., “Inflame and damage the cells that line the lungs: Within a few days, the damaged cells are replaced and the old cells are shed—much like the way your skin peels after sunburn. Studies suggest that if this happens repeatedly, lung tissue may become permanently scatted and lung function may be permanently reduced.”). Determine trigger details 2104 also may provide biophysical consequences of exposure to the determined trigger (e.g., “Make the lungs more susceptible to infection: Ozone reduces the lung's defenses by damaging the cells that move particles and bacteria out of the airways.”). Determine trigger details 2104 also may outline how the determined trigger affects other respiratory conditions (e.g., “Aggravated asthma: When ozone levels are unhealthy, more people with asthma have symptoms that require a doctor's attention or the use of medication. Ozone makes people more sensitive to allergens—the most common trigger for asthma attacks. Also, asthmatics may be more severely affected by reduced lung function and airway inflammation. People with asthma should ask their doctor for an asthma action plan and follow it carefully when ozone levels are unhealthy. Aggravate other chronic lung diseases: As concentration of ground-level ozone increase, more people with lung disease visit doctors or emergency rooms and are admitted to the hospital.”). Determine trigger details 2104 also may provide long-term effects of repeated exposure to the determined trigger (e.g., “Cause permanent lung damage: Repeated short-term ozone damage to children's developing lungs may lead to reduced lung function in adulthood. In adults, ozone exposure may accelerate the natural decline in lung function that occurs with age.”). In other examples, determine trigger details 2104 may include any other suitable information associated with the biophysical effects of exposure to the determined trigger. By displaying determined trigger view 2100, respiratory monitoring system 100 provides user 102 a notification of advice associate with at least one occurrence of a breathing event, at least one trigger, at least one trend, or the like. The notification of advice regarding the determined trigger may enable user 102 to improve control of occurrences of breathing events.

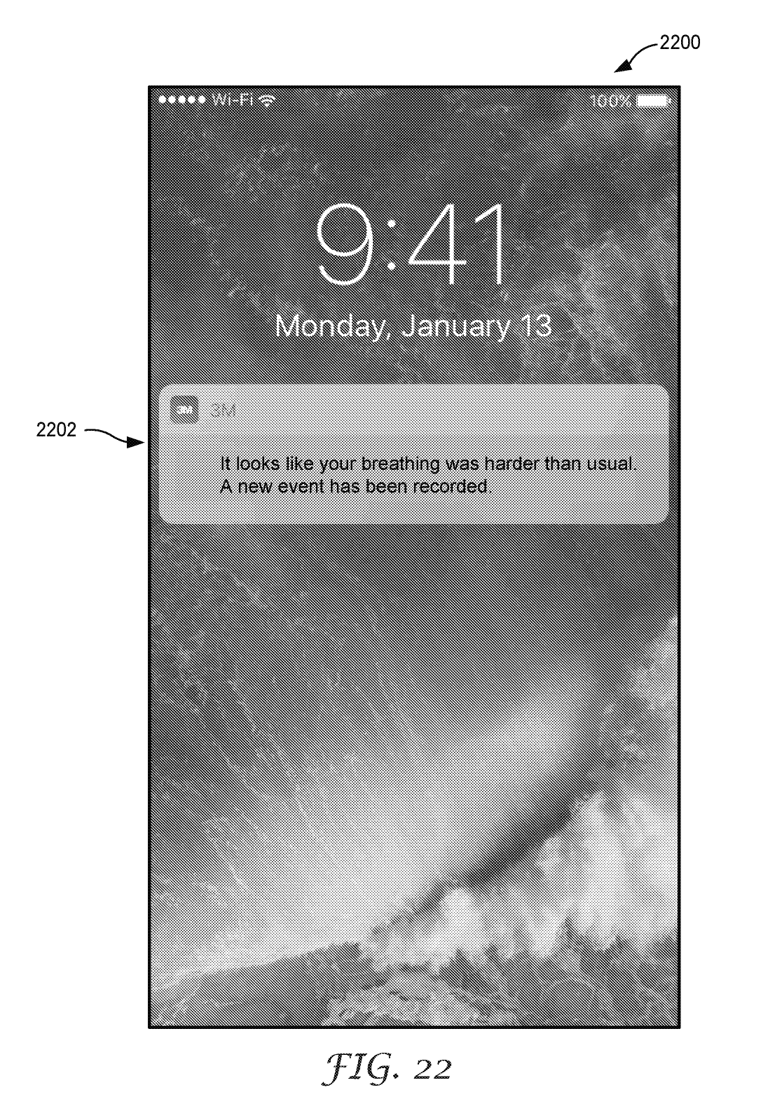

As discussed above, user interface 208 may receive from computing device 206 a notification includes at least one of an occurrence of a breathing event, a trigger, a trend, and advice, and cause display 210 to display the notification. FIG. 22 is a schematic and conceptual diagram illustrating an example push notification view 2200 displayed on a display 210 of respiration monitoring system 100. Push notification view 2200 includes push notification banner 2202. Computing device 206 may display push notification banner 2202 on the locked home screen of computing device 206. In some examples, the push notification may include a tactile alter (e.g., a vibration), an audible alter (e.g., a sound), or the like to notify user 102 of the push notification. As shown in push notification view 2200, the push notification banner 2202 includes an application logo (e.g., a blue square with white text displaying “3M”) and an application name (e.g., 3M). Push notification banner 2202 may also include a message indicative of the type of notification, such as, for example, an occurrence of a breathing event, a trigger, a trend, or advice. Generally, any suitable message may be displayed in push notification banner 2202. For example, push notification banner 2202 includes the following message: “It looks like your breathing was harder than usual. A new event has been recorded.”

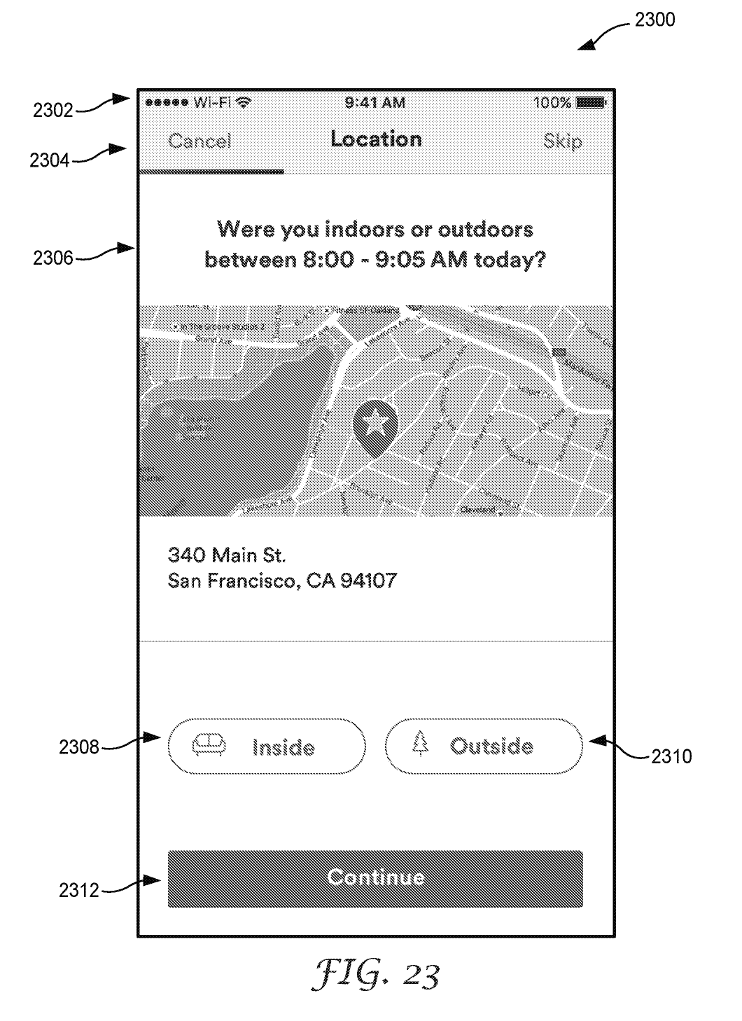

In some examples, in response to tapping the push notification banner 2202 and unlocking computing device 206, computing device 206 causes the display to display a survey that includes survey questions related to location information, environmental information, triggers associated with the occurrence of a breathing event, physical symptoms associated to the occurrence of a breathing event, or the like. For example, FIG. 23 is a schematic and conceptual diagram illustrating an example display of a location survey view 2300. Location survey view 2300 is related to location information associated with the occurrence of a breathing event. Location survey view 2300 includes status bar 2302, location survey navigator bar 2304, and location survey details 2306.

Status bar 2302 may be the same or substantially similar to status bar 902 of FIG. 9. Location survey navigator bar 2304 includes the title of the survey (e.g., Location) and selectable indicators to cancel the survey and skip to the next view of the survey. In other examples, location survey navigator bar 2304 may include other selectable indicators. Location survey details 2306 includes a survey question, an indication of a location of user 102 during the occurrence of the breathing event, survey buttons 2308 and 2310, and a continue survey button 2312. The survey question may include any suitable question to request user input regarding the location of user 102, such as, for example, “were you indoors or outdoors between 8:00-9:05 AM today?”. The indication of a location of user 102 may include, for example, a map showing an approximate location of user 102, an address, or both. Survey buttons 2308 and 2310 allow user 102 to provide the requested user input to answer the survey question. For example, survey button 2308 provide a selection for “inside,” whereas survey button 2310 provide a selection for “outside.” In response to selection of either one of survey buttons 2308 or 2310, computing device 206 receive input may allow user 102 to select the continue survey button 2312 to continue the survey. Selection of the continue survey button 2312 by user 102 causes computing device 206 to receive input representative of the location of the user during the occurrence of the breathing event and continue the survey.

In this way, computing device 206 may receive user input related to the location of user 102 during the occurrence of a breathing event. Computing device 206 may use the user input to adjust an algorithm used to determine at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like

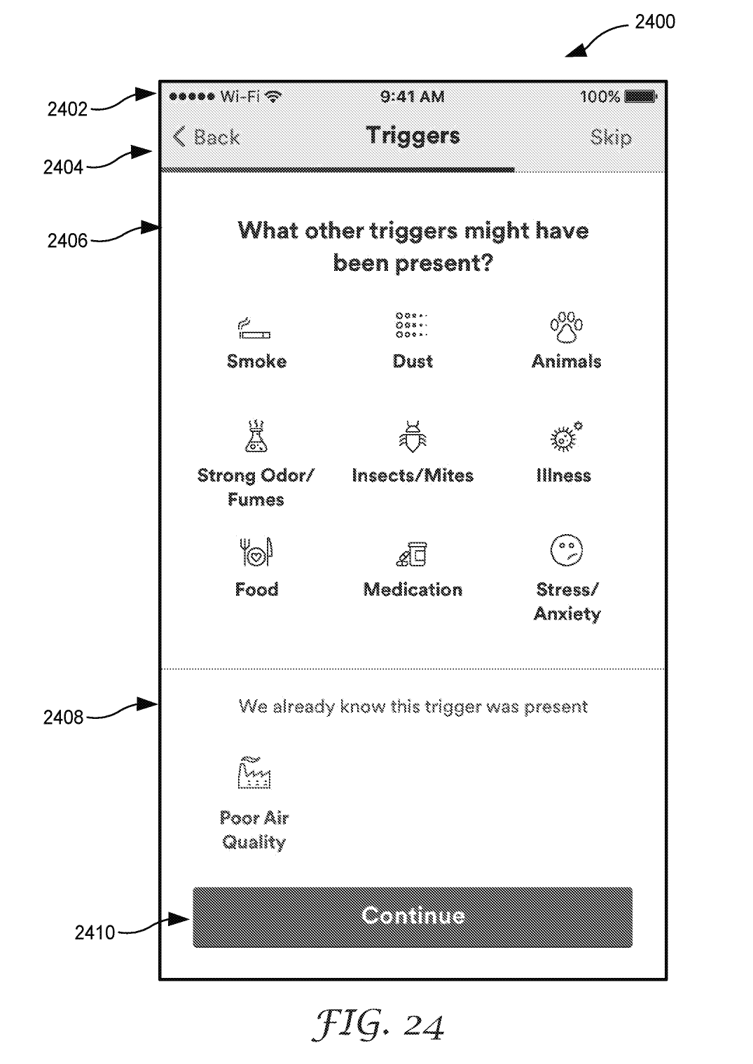

In response to user 102 selecting continue survey button 2312 or “skip” selectable indicator from location survey navigator bar 2304, computing device 206 may display a trigger survey view. For example, FIG. 24 is a schematic and conceptual diagram illustrating an example display of a trigger survey view 2400. Trigger survey view 2400 is related to triggers associated with the occurrence of a breathing event. Trigger survey view 2400 includes status bar 2402, trigger survey navigator bar 2404, and trigger survey details 2406.

Status bar 2402 and trigger survey navigator bar 2404 may be the same or substantially similar to status bar 2302 and location survey navigator bar 2304, respectively of FIG. 23, except for the differences described herein.

Trigger survey navigator bar 2404 includes the title of the survey (e.g., Triggers). Trigger survey details 2406 includes a survey question, an indication of potential triggers that may be associated the occurrence of the breathing event, an indication of known triggers 2408 that are associated the occurrence of the breathing event, and a continue survey button 2410. The survey question may include any suitable question to request user input regarding triggers that may be associated with the occurrence of the breathing event, such as, for example, “What other triggers might have been present?”. The indication of potential triggers may any potential trigger. For example, as shown in FIG. 24, the indication of potential triggers includes smoke, dust, animals, strong odor/fumes, insects/mites, illness, food, medication, and stress/anxiety. Each respective indication of potential triggers includes a selectable region that enables user 102 to select a respective potential trigger to provide user input that the selected potential trigger was present during the occurrence of the breathing event. Indication of known triggers 2408 includes any triggers that computing device 205 determined to be associated with the occurrence of the breathing event. For example, indication of known triggers 2408 includes poor air quality. In some examples, each respective indication of known triggers includes a selectable region that enables user 102 to select (or deselect) a respective known trigger to provide user input that the selected known trigger was not present during the occurrence of the breathing event. Selection of continue survey button 2312 by user 102 causes computing device 206 to receive input representative of the selected potential triggers and continue the survey.

In this way, computing device 206 may receive user input related to potential and known triggers present during the occurrence of a breathing event. Computing device 206 may use the user input to adjust an algorithm used to determine at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

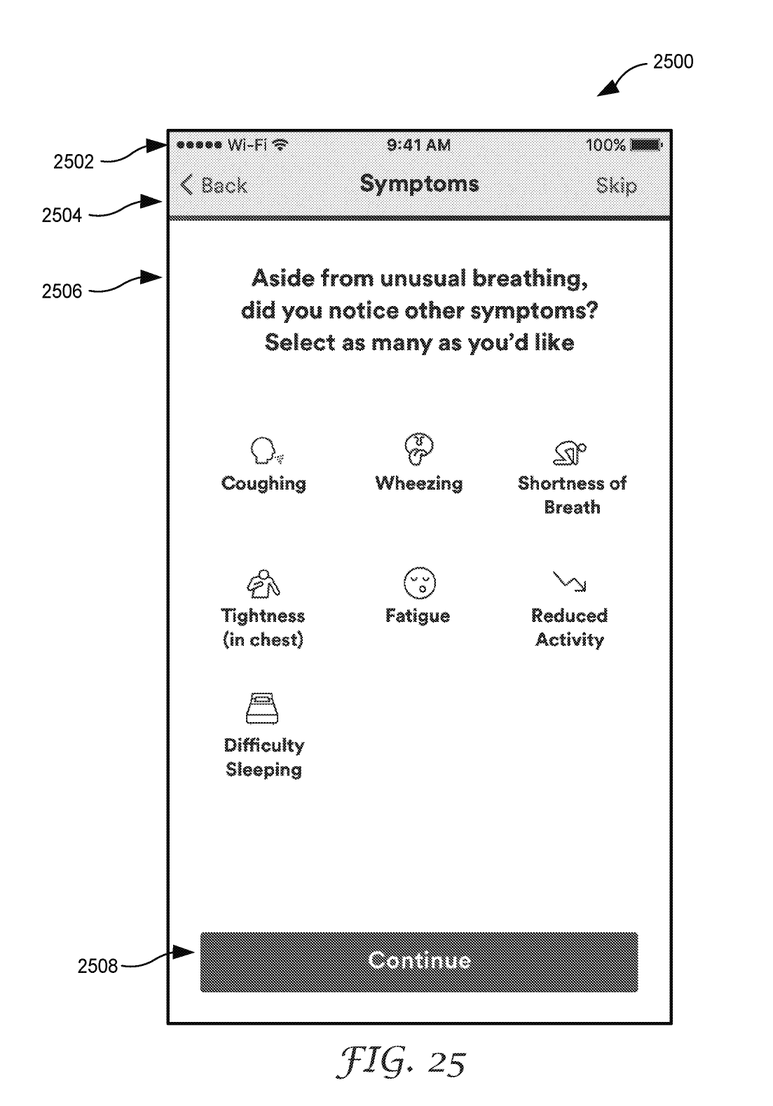

In response to user 102 selecting continue survey button 2410 or “skip” selectable indicator from trigger survey navigator bar 2404, computing device 206 may display a symptoms survey view. For example, FIG. 25 is a schematic and conceptual diagram illustrating an example display of a symptoms survey view 2500. Symptoms survey view 2500 is related to symptoms associated with the occurrence of a breathing event. Symptoms survey view 2500 includes status bar 2502, symptoms survey navigator bar 2504, and symptoms survey details 2506.

Status bar 2502 and symptoms survey navigator bar 2504 may be the same or substantially similar to status bar 2302 and location survey navigator bar 2304, respectively of FIG. 23, except for the differences described herein.

Symptoms survey navigator bar 2504 includes the title of the survey (e.g., Symptoms). Symptoms survey details 2506 includes a survey question, an indication of potential symptoms that may be associated the occurrence of the breathing event, and a continue survey button 2508. The survey question may include any suitable question to request user input regarding the symptoms user 102 experienced during the occurrence of the breathing event, such as, for example, “Aside from unusual breathing, did you notice other symptoms? Select as many as you′d like?”. The indication of potential symptoms may any potential symptom. For example, as shown in FIG. 25, the indication of potential symptoms includes coughing, wheezing, shortness of breath, tightness (in chest), fatigue, reduced activity, and difficulty sleeping. Each respective indication of potential symptoms includes a selectable region that enables user 102 to select a respective potential symptom to provide user input that the selected potential symptoms was present during the occurrence of the breathing event. Selection of continue survey button 2508 by user 102 causes computing device 206 to receive input representative of the selected potential symptoms and continue the survey.

In this way, computing device 206 may receive user input related to symptoms experienced by user 102 during the occurrence of a breathing event. Computing device 206 may use the user input to adjust an algorithm used to determine at least one of an occurrence of a breathing event, a trigger associated with at least one occurrence of a breathing event, a trend associated with at least one occurrence of a breathing or at least one trigger, advice associate with at least one occurrence of a breathing event, at least one trigger, at least one trend, or the like.

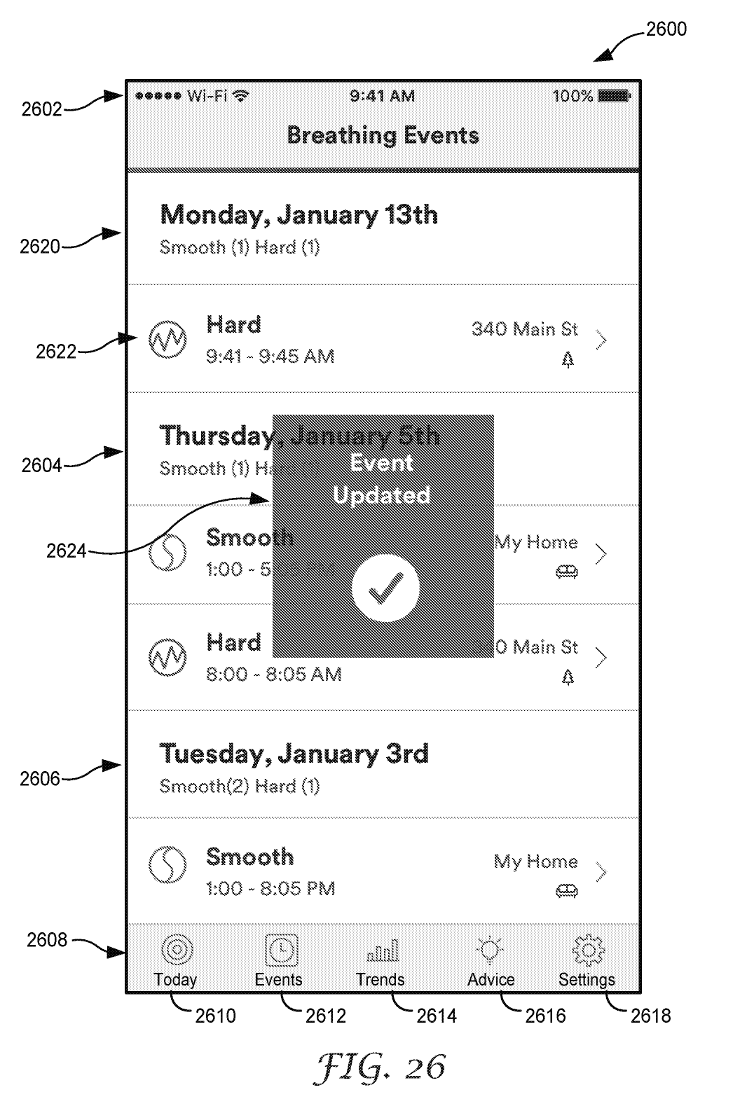

In response to user 102 selecting continue survey button 2508 or “skip” selectable indicator from trigger survey navigator bar 2504, computing device 206 may display an event updated notification. For example, FIG. 26 is a schematic and conceptual diagram illustrating an example display of an event updated notification view 2600. Event update notification view 2500 may be the same or substantially similar to the breathing events view 1400 discussed above with respect to FIG. 14, except for the differences described herein. For example, like breathing events view 1400, breathing events view 2600 is titled “Breathing Events,” and includes a status bar 2602, a first list of breathing events 2604, a second list of breathing events 2606, and navigation bar 2608 that includes five navigation tiles (e.g., Today 2610, Events 2612, Trends 2614, Advice 2616, and Settings 2618).

As shown in FIG. 26, breathing events view 2600 includes a third list of breathing events 2620. Third list of breathing events 2602 includes new event 2622 (e.g., an updated event). New event 2622 includes a date (e.g., Monday, January 13th) and an indication of the number of smooth breathing events and hard breathing event that occurred on that date (e.g., Smooth (0), Hard (1)). New event 2622 includes a graphic and indicator of the type of breathing event (e.g., a hard breathing event), a duration or timeframe of the occurrence of the breathing event (e.g., 9:41-9:45 AM), and a location of the breathing event (340 Main St.). Additionally, breathing events view 2600 includes notification 2624 indicating that the event was updated. By displaying new event 2622 in breathing events view 2600, respiratory monitoring system 100 provides user 102 a notification that new breathing event 2600 was recorded.

An example system for respiratory monitoring may include: a sensor, where the sensor monitors at least one respiratory related parameter; a computing device connected to the sensor, where the computing device includes a processor, where the processor detects a first breathing state based on the at least one respiratory related parameter received from the sensor; a user interface, where the user interface receives user input related to the first breathing state, and where the processor modifies the detection of one or more breathing states based on the user input.

An example non-transitory computer-readable storage medium that stores computer system executable instructions that, when executed, may configure a processor to: detect a first breathing state based on at least one respiratory related parameter received from a sensor, where the sensor monitors the at least one respiratory related parameter; and modify the detection of one or more breathing states based on user input related to the first breathing event, where a user interface receives the user input.

Various examples have been described. These and other examples are within the scope of the following claims.

1. A method for respiratory monitoring, comprising:
receiving, by a computing device, a feature vector having a plurality of features, each respective feature associated with a respective respiratory related parameter;
receiving, from a sensor, at least one respiratory related parameter associated with a respective feature of the feature vector; and
detecting, by the computing device, a first breathing state based on the at least one respiratory related parameter received from the sensor.

2. The method of claim 1, wherein detecting the first breathing state comprises applying, to each of the at least one respiratory related parameters received from the sensor, a first weight assigned to the feature associated with the respective respiratory related parameter received from the sensor.

3. The method of claim 2, further comprising detecting, by the computing device, a second breathing state based on applying, to each of the at least one respiratory related parameters received from the sensor, a second weight assigned to the feature associated with the respective respiratory related parameter received from the sensor.

4. The method of claim 2, wherein the method further comprises:
displaying, on a display communicatively coupled to the computing device, an indication that the first breathing state was detected;
receiving, from a user interface, user input related to detecting of the first breathing state; and
adjusting one or more of the first weights in view of the user input.

5-6. (canceled)

7. The method of claim 1, further comprising receiving, by the computing device, at least one second respiratory related parameter associated with a feature of the feature vector, wherein the at least one second respiratory related parameter includes information selected from at least one of environmental information, location information, and user input.

8. The method of claim 7, wherein detecting the first breathing state comprises applying, to each of the at least one respiratory related parameters received from the sensor, a first weight assigned to the feature associated with the respective respiratory related parameter received from the sensor and applying, to each of the at least one second respiratory related parameters received, a first weight assigned to the feature associated with the respective second respiratory related parameter.

9. The method of claim 7, wherein the environmental information comprises weather information and air quality information, and wherein the user input comprises respiratory related symptoms.

10. The method of claim 7, further comprising displaying, on a display communicatively coupled to the computing device, a trigger detected based, at least in part, on the at least one second respiratory related parameter.

11. The method of claim 10, further comprising:
receiving, by the computing device, user input related to detecting the trigger; and
modifying the detecting of the trigger based on the user input.

12. The method of claim 3, further comprising displaying, on a display communicatively coupled to the computing device, a trend determined based on one or more of the first and second breathing states and at least one second respiratory related parameter associated with a feature of the feature vector, wherein the at least one second respiratory related parameter includes information selected from at least one of environmental information, location information, and user input.

13. The method of claim 12, further comprising:
receiving, by the computing device, user input related to the trend; and
modifying the detecting of the trend based on the user input.

14. The method of claim 1, wherein the method further comprises:
displaying, on a display communicatively coupled to the computing device, an indication that the first breathing state was detected; and
displaying, on the display, advice determined by the computing device to be relevant to the first breathing state.

15. The method of claim 14, further comprising:
receiving, by the computing device, user input related to the advice; and
modifying the advice provided with the first breathing state based on the user input.

16-31. (canceled)

32. A system for respiratory monitoring, comprising:
a sensor, wherein the sensor monitors at least one respiratory related parameter; and
a computing device connected to the sensor,
wherein the computing device includes a processor,
wherein the processor receives a feature vector having a plurality of features, each respective feature associated with a respective respiratory related parameter,
wherein the processor receives from the sensor the at least one respective respiratory related parameter, and
wherein the processor determines a first breathing state based on the feature vector.

33. The system of claim 32, wherein the processor applies, to each of the at least one respiratory related parameters received from the sensor, a first weight assigned to the feature associated with the respective respiratory related parameter received from the sensor.

34. The system of claim 33, wherein the processor determines a second breathing state based on applying, to each of the at least one respiratory related parameters received from the sensor, a second weight assigned to the feature associated with the respective respiratory related parameter received from the sensor.

35. The system of claim 33, further comprising:
a display connected to the computing device, wherein the processor causes the display to display an indication of the first breathing state; and
a user interface connected to the computing device, wherein the user interface to received user input related to the first breathing state,
wherein the processor adjusts one or more of the first weights based on the user input.

36. The system of claim 32, wherein the first breathing state is a hard breathing state.

37. The system of claim 32, wherein the at least one respiratory parameter comprises parameters associated with inhale activities and parameters associated with exhale activities.

38. The system of claim 32, wherein the processor receives at least one second respiratory related parameter associated with a feature of the feature vector, wherein the at least one second respiratory related parameter includes information selected from at least one of environmental information, location information, and user input.

39. The system of claim 38, wherein the processor applies, to each of the at least one respiratory related parameters received from the sensor, a first weight assigned to the feature associated with the respective respiratory related parameter received from the sensor and applies, to each of the at least one second respiratory related parameters received, a first weight assigned to the feature associated with the respective second respiratory related parameter, to determine the first breathing state.

40. The system of claim 38, wherein the environmental information comprises weather information and air quality information, and wherein the user input comprises respiratory related symptoms.

41. The system of claim 38, wherein the processor determines, and causes the display to a display, a trigger based, at least in part, on the at least one second respiratory related parameter.

42. The system of claim 41, wherein the processor receives user input related to the trigger and modifies the trigger based on the user input.

43. The system of claim 34, wherein the processor determines, and causes the display to a display, a trend based, at least in part, on one or more of the first and second breathing states and at least one second respiratory related parameter associated with a feature of the feature vector, wherein the at least one second respiratory related parameter includes information selected from at least one of environmental information, location information, and user input.

44. The system of claim 43, wherein the processor receives user input related to the trend and modifies the trend based on the user input.

45. The system of claim 32, wherein the processor determines, and causes the display to display, advice based on the first breath state.

46. The system of claim 45, wherein the processor receives user input related to the advice and modifies the advice based on the user input.

47-60. (canceled)
